(19) United States
(12) Patent Application Publication
(10) Pub. No.: US 2021/0393558 A1
(43) Pub. Date: Dec. 23, 2021

(54) METHODS AND COMPOSITIONS FOR PREVENTION AND TREATMENT OF PRESSURE ULCERS

(71) Applicants: TECHNION RESEARCH AND DEVELOPMENT FOUNDATION LTD., Haifa (IL); RAMOT AT TEL-AVIV UNIVERSITY LTD., Tel-Aviv (IL)

(72) Inventors: Daphne Weihs, Haifa (IL); Amit Gefen, Ganei Tikva (IL)

(21) Appl. No.: 16/957,491

(22) PCT Filed: Dec. 31, 2018
(86) PCT No.: PCT/IL2018/051424

§ 371 Date: Jun. 24, 2020

(30) Foreign Application Priority Data
Dec. 31, 2017 (IL) 256675

(51) Int. Cl. A61K 31/19; A61F 5/32; A61F 13/02; A61K 31/22; A61L 15/44

(52) U.S. Cl. CPC A61K 31/19; A61F 5/32; A61F 2013/00404; A61K 31/22; A61L 15/44; A61F 13/023

(57) ABSTRACT

The technology subject of the present application aims at providing a methodology for prevention of pressure ulcers.

### TECHNOLOGICAL FIELD

The invention generally concerns methods and compositions for preventing and treating pressure ulcers and injuries.

### BACKGROUND

Pressure ulcers (PUs) also termed bedsores, decubitus ulcers, pressure sores or pressure injuries are a common and serious health issue that may form at the skin, and/or at deeper tissues (e.g. muscles), and then may spread from deeper tissues towards the skin or vice versa. The formed PUs may span from skin to underlying tissues including but not limited to fat, tendon, fascia and muscle and may affect bones.

A PU may form due to sustained bodyweight forces in subjects that are under temporary or permanent sensory impairment and/or muscular conditions limiting mobility. Individuals who are at risk are persons confined to bed, chair-hound or wheelchair users, persons under anesthesia (e.g. surgical patients), those who cannot respond to or report inconvenience or pain, or individuals who are otherwise healthy but at a certain time are unconscious or under analgesic or other medications impairing sensation and/or movement. The problem is also seen in skin-contact devices or objects that impose sustained pressure onto the skin and underlying tissues. PUs can be observed in patients of all ages, health conditions, and in a variety of health care facilities, e.g. hospitals, clinics, long-term care facilities, community care facilities and homes. For many patients, particularly the elderly and those with central nervous system injuries or neuromuscular conditions, PUs may inexplicably become chronic and remain so for the remainder of the patient's lifetime. Many serious PUs (category 3 and 4) become chronic wounds, can easily deteriorate and can lead to mortality from complications, e.g. sepsis, osteomyelitis, renal or organ system failure.

Current prevention strategy is focused on routine skin assessments, repositioning, prescription of specialized support surfaces and in some countries prophylactic dressings to redistribute soft tissue loads, as well as nutritional interventions in at-risk patients. Technologies for monitoring early signs of soft tissue damage where the condition may still be reversible, such as detection of localized edema (sub-epidermal moisture) using bioimpedance are also penetrating the market. Nevertheless, the prevalence of PUs remains unacceptably high across modern care facilities in developed countries, e.g. at 20-30% in geriatric settings and around 10% for surgical patients.

PUs have recently been shown to be caused primarily by sustained cell and tissue deformations. Large sustained soft tissue deformations lead to initiation of cell necrosis through progressive loss of integrity of the cytoskeleton and poration of the plasma membrane. The sustained deformations also hamper proper repair of the early cell damage, when it is still reversible, as perfusion, lymphatic drainage and (immune) cell migration capacities are all impaired. Gradually, ischemia builds up which reduces delivery of metabolites and clearance of waste products, and further adds biochemical stress to the affected cells as the lactate concentration increases and the pH level drops. Hypo- or hyper-inflammation response which are common in at-risk individuals such as the elderly and those with diabetes may amplify cell and tissue damage as well. The damage pathway can progress from the surface of the body (i.e. the skin) inwards, as in superficial PUs, but the more severe injuries appear to progress from within deep tissues (e.g. skeletal muscle or subcutaneous fat) outwards, and are then termed deep tissue injury (DTI). A combination of inside-out and outside-in damage progression is also possible. At the cell level, exposure to deformation compromises homeostasis (i.e. biological equilibrium) as a result of the uncontrolled molecular and ion transport inwards to the intracellular space and outwards to the extracellular environment across the damaged plasma membrane [1-4].

Microclimate factors such as the build-up of heat at the body-support interface, the related skin and subcutaneous tissue temperatures, and the extent of accumulation of moisture and wetness on the skin are considered important factors in the etiology of PUs as well. Tissue temperature affect the metabolic rate (vascular supply and cellular demand) and hence influence the tolerance to of tissues ischemic events. Moisture and wetness affect the skin micro-topography which in turn affects the friction coefficient of skin in interaction with other materials and the delivered frictional forces that distort skin and deeper tissues. At the same time, moisture and wetness affect the stiffness and strength of the skin, generally making the skin more fragile and prone to mechanical injury.

The mechanism leading to the development of PUs is not subject-dependent. PUs can develop in any individual, with or without an underlying medical condition, if they are incapable of avoiding prolonged periods of an uninterrupted tissue distortion, primarily from pressure and shear as a result of bodyweight forces or forces from an external device which contacts the skin. PUs are progressive in nature and most frequently found in bedridden, chair-bound or immobile people, particularly if sensation of discomfort and pain are impaired e.g., due to a neurological injury or disease, peripheral neuropathy, sedation or anesthesia. They often develop in people who have been hospitalized for a long time generally for a different problem and increase the overall time as well as cost of hospitalization that have detrimental effects on patient's quality of life. Loss of sensation compounds the problem manifold, and failure of reactive hyperemia cycle of the pressure prone area remains the most important etiopathology [10].

Many of the patients affected with PUs frequently develop it over a bony prominence, wherein the majority of the cases are affected over the area where skin covers bones such as sacral, ischial and trochanteric pressure ulcers [11] and the lower extremities as observed in the malleolar, heel, patellar and pretibial locations, account for approximately 25% of all pressure sores [12]. Other common PUs are associated with the use of medical devices for diagnostics or treatment, and such PUs typically conform the shape of the device or develop at contact areas of the device with the body e.g. the bridge of the nose, cheeks or chin when using continuous positive airway pressure (CPAP) masks. Other examples are PUs caused by use of catheters, endotracheal and nasogastric tubing, ECG/EEG electrodes or a pulse oximeter which apply localized sustained deformations on the interfacing skin and subdermal tissues, either when placed properly or, sometimes, when they are misplaced as a result of a human error. PUs associated with use of medical devices which develop in the face and head may later on have serious psychological implications and lead to body image issues, in children as well as in adults. Heel PUs often lead to limb loss and are common in patients with diabetic neuropathy. In fact, diabetic nonhealing wounds are the leading cause of nontraumatic amputations in the United States [13].

As explained above, another important type of PUs is medical device-related PUs, as the typical hospital bed, particularly in emergency, trauma, intensive care and surgical departments would often contain catheters and tubing, ECG electrodes, a cuff of a blood pressure monitor, a pulse oximeter, wiring, etc. On the bed, potentially, there could be forgotten small objects including plastics (e.g. needle covers), consumables or pieces of packaging of sterile equipment which may have unnoticeably fallen or have been forgotten on the bed, and may become in contact with the patient. Rigid equipment, devices and any objects that are misplaced between the body and support surface act like an ‘external bony prominence’ and inflict localized, aggressive soft tissue distortions in addition to those caused near the bony prominences due to bodyweight. Importantly, medical device-related PUs can also be caused by devices that have been specifically designed to be in continuous contact with the body tissues, particularly CPAP masks which are again a common type of equipment used in the aforementioned medical settings. In particular, CPAP masks often cause PUs at the bridge of the nose due to the continuous mechanical loading that they apply. Likewise, it is typical to find hospital-acquired PUs on the (lower) lips of intubated patients, or on the nostrils of patients fed with nasogastric tubes.

Currently, PU prevention strategies are mostly based on macro-scale interactions of the patient body with support surfaces or surface of pressure inducing devices. Traditionally, the focus was on mattresses and (wheelchair) cushions [5-8]. Recently, prophylactic (prevention) dressings were introduced in the market. Such products are aimed at cushioning vulnerable body sites and alleviating mechanical loads from soft tissues through mechanisms such as cushioning to redistribute pressures, absorbance of internal shear in the dressing, and minimization of frictional forces by means of low coefficient of friction of external dressing surfaces, as well as microclimate management [9]. Low-friction fabrics have also been introduced in the market for producing garments and bootees to protect the sacral and heel areas from elevated frictional forces.

There are various types of treatments for PUs that aim to reverse the factors that have originally caused the initial tissue damage or the full-scale PU. Some commonly used treatments are provided by Bhattacharya and Mishra [14] and include nutrition supplements (e.g. protein, iron, Vitamin-C and zinc), cleaning of the wound and maintaining meticulous skin care, removal of dead tissue using low-frequency ultrasound energy waves or using laser beams of light, wound dressing specialized for every stage of the PU, prophylactic dressings; application of hydrocolloid gel—a special gel that encourages the growth of new skin cells in the ulcer and keeps the nearby healthy area of skin dry, alginate dressing that speed up the healing process, nano-silver dressing that use the antibacterial property of silver to clean the ulcer; polymeric membrane dressings with material composition that manages tissue inflammation and contains it to the acute site of the injury; dressing with dilute ascetic acid, various creams and ointments to prevent further tissue damage and help speed up the healing process; antibiotics for treating infected PUs and preventing the infection from spreading and support surfaces (including mattresses and cushions).

U.S. Pat. No. 5,658,956 [15] discloses therapeutic bioadhesive-wound healing compositions comprising (a) pyruvate; (b) an antioxidant; and (c) a mixture of saturated and unsaturated fatty acids for treating wounds (e.g. bed sores, pressure ulcers, cutaneous, decubitus, venous stasis, and diabetic ulcers), and increasing the proliferation and resuscitation rate of mammalian cells. The bioadhesive-wound healing composition may be utilized in a wide variety of pharmaceutical products. This invention also relates to methods for preparing and using the bioadhesive-wound healing compositions and the pharmaceutical products in which the compositions may be used.

U.S. Pat. No. 5,602,183 [16] proposes therapeutic dermatological-wound healing compositions useful to minimize and treat diaper dermatitis. The compositions comprise a pyruvate material along with antioxidants and mixtures of saturated and unsaturated fatty acids.

US application no. 2005/197397 [17] proposes methods for treating wounds and diseases in mammals, caused by mammalian cells involved in an inflammatory response, by altering indigenous in vivo levels of peroxynitrous acid, and salts thereof. The method comprises contacting the mammalian cells with a therapeutically effective amount of a reactive oxygen species mediator, wherein the reactive oxygen species mediator is selected from the group consisting of pyruvates, pyruvate precursors, a-keto acids having four or more carbon atoms, precursors of a-keto acids having four or more carbon atoms, and the salts thereof.

### REFERENCES

[1] Leopold E and Gefen A. Med Eng Phys. 35(5):601-607 (2013).
[2] Slomka N and Gefen A. Ann Biomed Eng. 40(3):606-618 (2012).
[3] Slomka N, et al., Cell. Moll. Bioeng 2:118-132 (2009).
[4] Gefen A and Weihs D, Med Eng Phys. 38(9):828-833 (2016).
[5] Levy A, et al., J. Rehabili. Res. Dev 51:1297-1319 (2014).
[6] Levy A, et al., J. Tissue Viability. 23:13-23 (2014).
[7] Levy A, et al., J. Mech. Behay. Biomed. Mater. 28:436-447 (2013).
[8] Levy A, et al., Adv. Wound Care 4(10): 615-622 (2015).
[9] Levy A, Schwartz D, Gefen A. Int Wound J. 14(6):1370-1377 (2017).
[10] Gebhardt K S. Nurs Times. 98:4 (2002).
[11] Vasconez L O, et al. Curr Prob Surg. 62:1-62 (1977).
[12] Cannon B C and Cannon J P. Am J Health Syst Pharm. 61:1895-1905 (2004).
[13] American Diabetes Association. Diabetes Care. 36(4):1033-1046 (2013).
[14] Bhattacharya and Mishra. Indian J Plast Surg. 48(1): 4-16 (2015).
[15] U.S. Pat. No. 5,658,956.
[16] U.S. Pat. No. 5,602,183.
[17] US application no. 2005/197397.

### SUMMARY OF THE INVENTION

The mechanism of pressure ulcers (PUs) formation is a complex process with numerous physiological and pathophysiological factors that are specific to the individual, hence the difficulties in predicting the occurrence of and preventing the development of PUs. The inventors of the invention disclosed herein have developed a simple and straight-forward method to preventing PUs by reducing the effect or arresting development of an early phase of damage, if such has developed. The herein described method can be used on its own or as complementary to existing prevention and treatment technologies or methodologies (e.g. dressings, padding, support surfaces, garments, diapers, surfaces of medical devices that apply forces and cause sustained tissue deformations).

Unlike existing methodologies for preventing PUs that focus on better pressure distributing surfaces and absorbance of deformation energy in the device, so that tissue deformations are alleviated (e.g. static or dynamic mattresses, support surfaces made of smart materials with memory shape and prophylactic dressings), which do not eliminate the risk for tissue breakdown completely, the method of the present invention enables prevention of micro-damage associated with PUs and provides protection against cell damage at a very early stage in the development of PUs. Thereby, the present method addresses the problem already at a cellular scale (as opposed to a macroscopic scale) and treat the early micro-damage at the cellular scale, hence supporting the body physiological mechanisms in doing so. The present method can save lives and improve the quality of life of billions of patients worldwide at surgical and acute care, elderly and nursing home settings as well as in community medicine and home-based treatments, and hence revolutionizes cost-benefit models in PU prevention.

The methodology disclosed herein does not concern treatment of already existing PUs that are visible to the naked eye, nor the treatment of tissue damage where the origin may not be directly associated with the development of PUs, but which may be eventually diagnosed or treated as such. To the contrary; the present invention provides a methodology involving local administration of a pyruvate compound to prevent, arrest, ameliorate, or treat PUs at their earliest stage of cell-level damage, thereby, preventing deterioration to the tissue and systemic scales and complications associated therewith. Importantly, without being bound by theory, methods of the present invention improve the efficacy of treatment by preventing development of PUs at an early stage—where damage is still microscopic and very likely reversible, thereby allowing patients to undergo diagnostics or treatment. For example, the invention enhances cell tolerance to distortion in imaging investigations (e.g. MRIs), vascular catheterization, endoscopy or arthroscopy, epidural anesthesia, surgical and recovery procedures requiring anesthesia or sedation, use of medical devices such as CPAP masks, spine (back) boards, endotracheal and nasogastric tubes, or other devices for prolonged periods and any other scenario where bodyweight or external forces continuously distort soft tissues and the body is unable to respond and relief the mechanical loads in the soft tissues.

In other words, the present invention aims at providing a methodology for prevention of PUs at a stage where PUs are not visible to the naked eye as the damage is still microscopic, at the cell scale, hence would not be detectable for example by using standard medical imaging such as ultrasound or during routine visual assessments. In those stages, the development of PU is prevented, arrested, repaired on the microscale or otherwise controlled so that it does not deteriorate further into a macroscopic, clinically significant tissue damage.

For achieving the main purposes of the invention, a formulation comprising at least one pyruvate material, that need not comprise any other active material, has been devised to be administered optionally with a low- or medium-level stretching (under strains where major cell damage does not occur). In some embodiments of the present invention, a formulation or an article according to the invention consists, as an active material and method for preventing PU, at least one pyruvate that may or may not be combined with low- or medium-level stretching. In other cases, a formulation or an article of the invention is free of an antioxidant material.

In a first aspect, the invention provides a method for preventing and/or treating early stage PU in a subject, comprising locally administering a formulation comprising or consisting an effective amount of a pyruvate compound or at least one pharmaceutically acceptable salt thereof to a skin region or to an organ of the subject who has demonstrated at least one characteristic of an early stage PU or has predisposition to suffer from or susceptible to suffering from a PU and is thus at risk of developing PU.

In some embodiments, the method comprises the following stages:

(a) Measuring level or amount of at least one inflammatory biomarker from at least one tissue region of a subject susceptible to develop a PU. Erythema or redness of the skin, temperature changes, texture abnormalities and presence of edema are clinical symptoms of the inflammatory response. In addition, quantitative objective measurements based on biophysical or biochemical markers can provide alerts regarding the onset and progression of an inflammatory response.
An example for a biophysical biomarker indicative of a subdermal inflammatory response is measurements by means of SEM Scanner. The SEM Scanner identifies early, deformation-inflicted tissue damage, by detecting changes in subepidermal moisture (SEM) which is the localized Edema that occurs early during the inflammatory process. The SEM Scanner is able to detect micro-scale edema 3-10 days before a PU is visible to the naked eye. The SEM Scanner technology is described in more detail in the section “Measuring subepidermal moisture to detect pressure injury” where the relevant literature demonstrating the early-detection capacity sis cited.
Examples for biochemical biomarkers indicative of an inflammatory response in skin are elevated cutaneous interleukin 1α (IL-1α) and lactate concentrations that can be assessed using a commercial, established Sebutape-based technology (Hemmes B, de Wert L A, Brink P R G, Oomens C W J, Bader D L, Poeze M. Cytokine IL1α and lactate as markers for tissue damage in spineboard immobilisation. A prospective, randomised open-label crossover trial. J Mech Behav Biomed Mater, 2017; 75:82-88). Examples for biochemical biomarkers indicative of an inflammatory response in adipose tissue include Hmox1, interleukin-6, interleukin-1, and monocyte chemoattractant protein-1 (Gust M J, Hong S J, Fang R C, Lanier S T, Buck D W 2nd, Nuñez J M, Jia S, Park E D, Galiano R D, Mustoe T A. Adipose Tissue Drives Response to Ischemia-Reperfusion Injury in a Murine Pressure Sore Model. Plast Reconstr Surg. 2017; 139(5):1128e-1138e). Examples for biochemical biomarkers indicative of an inflammatory response in skeletal muscle (typical in deep tissue injuries) are abnormally increased levels of myoglobin (Mb), heart-type fatty acid binding protein (H-FABP), and C-reactive protein (CRP, Loerakker S, Huisman E S, Seelen H A, Glatz J F, Baaijens F P, Oomens CW, Bader DL. Plasma variations of biomarkers for muscle damage in male nondisabled and spinal cord injured subjects. J Rehabil Res Dev. 2012; 49(3):361-72).

(b) Comparing said level or amount with a reference level or amount of said biomarker, obtained from a population that is not afflicted with PU and determining the presence or the risk of developing early stage PU with high specificity based on said comparison;

(c) Locally administering a formulation comprising or consisting an effective amount of a pyruvate compound or at least one pharmaceutically acceptable salt thereof to the skin region or to an organ of the subject who is at risk of or showing signs of early stage PU.

The “skin region” referred to in the present disclosure is the skin area where a PU has initiated or that is near to, or above an affected deeper tissue, or included in the PU-damaged tissues, or is susceptible to PU or is at-risk for a PU. Any such “skin region” is referred to in the present disclosure as the skin region of interest where any embodiments of the invention are applied.

The term “pressure ulcer” (interchangeable herein with “pressure injury” bedsore, pressure sore and decubitus ulcer) is well known in the art. For example, a definition of the term “pressure injury” is provided by the National Pressure Ulcer Advisory Panel (NPUAP), as a localized damage to the skin and/or underlying soft tissue usually over a bony prominence or related to a medical or other device, resulting from an intense and/or prolonged pressure or pressure in combination with shear. The injury can present as intact skin or an open ulcer and may be painful. The injury is not any injury to a skin region or a tissue that is not caused due to an intense and/or prolonged pressure or pressure in combination with shear.

The “early stage PU” generally refers to a stage that precedes a condition commonly recognized as a Grade I ulcer, being the first stage of superficial ulcers in the skin and is classified as presenting at least one of the following non-blanchable erythema intact skin, discoloration of the skin, warmth and edema. Induration or hardness may also be used as indicators, particularly on individuals with darker skin. Early stage PU does not involve macroscale inflammation at the body site susceptible to develop PU.

Early stage PU may be detected by various medical technologies (e.g. transdermal analysis patch (TAP); elevated sub-epidermal moisture (SEM)) which can measure changes in the skin that can predict development of PUs.

The early stage of tissue damage may thus be characterized by an increased level of at least one inflammatory biomarker in at least one region of the body susceptible to developing PUs. The said increased level is generally measured through variability of measurements at several locations at and near the potential site for PU development, e.g. points around the sacrum, starting at times prior to imposing conditions that may cause the development of PUs. Microscopic damage will initially be localized at one location and thus early indication will be when one location abnormally differs from others. For example, using the SEM Scanner technology to detect a rise in tissue biocapacitance associated with microscopic edema, a set of readings is performed on each anatomical site under assessment, the readings being at distances e.g. of 2-5 cm from each other (as per manufacturer guidelines). After the set of readings have been collected for the assessment, a delta (A) is calculated between the maximal and minimal SEM reading values, to indicate variability in tissue health status across the site-associated readings. The inflammation may be generally characterized by infiltration of immune system cells e.g. neutrophils to the damage site and/or build-up of edema at the site. Detection of localized inflammatory response is feasible and measurable by existing medical technologies, as further detailed herein.

The biomarkers may be any protein (e.g. cytokines, interleukins) which level can be used to measure, assess and/or determine the development of early stage PU. In some embodiments, the inflammatory biomarkers can be, for example, selected from IL-1α; IL-1β; IL-1; IL-6; IL-1RA; TNF-α; IFN-γ; CCL-1; VEGF; thrombospondin-1 and -2; anti-microbial peptides (e.g. β-Defensin-1 (hBD-1) and -2 (hBD-2), or similar biomarkers that are indicative of damage formation. For example, using TAP, inflammatory biomarkers are measured from the skin of bed-ridden patients susceptible to develop PUs and treatment with the formulation of the invention is initiated only when the level or amount of the biomarkers (e.g. IL-1α, IL-1RA, CXCL-1/2, human β-defensin-1) is indicative of early stage PU.

According to this method, for example, TAP capture antibody micro-arrays coated with various antibodies (e.g. anti-IL-1α, anti-IL-1RA, anti-CXCL-1/2 and anti-hBD-1) are applied (e.g. in duplicates) to the inside part of the body susceptible to develop a PU (e.g. lower back), wetted on the skin (e.g. with PBS) and incubated. Following incubation, TAP capture antibody micro-arrays are collected from the skin and stored (e.g. at 4° C.) until further analysis (e.g. using spot-ELISA).

Additional biomarkers and assays that may be used in accordance with the invention may be found in one or more of the following: (a) Orro K, Smirnova O, Arshayskaja J, et al. Development of TAP, a non-invasive test for qualitative and quantitative measurements of biomarkers from the skin surface. Biomarker Research. 2014; 2:20. (b) Gefen A. Managing Inflammation by Means of Polymeric Membrane Dressings in Pressure Ulcer Prevention. Wounds International Vol. 9, pp. 22-28, 2018. (c) Gefen A. The Sub-Epidermal Moisture Scanner: The Principles of Pressure Injury Prevention using Novel Early Detection Technology. Wounds International, Vol. 9, pp. 10-15, 2018. (d) Gefen A, Gershon S. Sub-epidermal moisture measurements for early detection of pressure injuries compared to ultrasound and visual skin assessments: An observational, prospective cohort pilot study. Ostomy Wound Management, Vol. 64(9), pp. 12-27, 2018.

Within the context of the present invention, the term “early stage PU” also encompasses microscopic damage to cells, tissues, skin, fat, muscle, tendons, connective tissues, other tissues or organs in a subject's body that cascades the development of visible pressure injuries as defined. The prevention of these microscopic damages by methods of the invention permits arresting or minimizing the development of pressure injuries. Further, treatment of these microscopic damages which may prevent their evolution or development into pressures injuries is also within the scope of the present invention.

At a microscopic scale, pressure injuries onset as the first cells die due to loss of homeostasis caused by uncontrolled transport of biomolecules and ions through their plasma membrane. The loss of homeostasis occurs as the cytoskeleton of the distorted cells is damaged, followed by poration of the plasma membrane which is structurally supported by the (failing) cytoskeleton. Poration of the cells and loss of homeostasis leads to death of the cell and sends damage signals to neighboring cells. These cell death events trigger a necrosis-apoptosis damage spiral that may become en masse within several minutes.

Specialized test devices designed to subject cell cultures to controlled, sustained deformations were used to determine plasma membrane permeability in externally deformed cells (at damaging levels) by means of fluorescent dyes. Fluorescent dyes that are too large to enter an intact plasma membrane can be tracked as they enter the damaged cell bodies from the culture media where they have been introduced. It has been shown that under sustained and high, though physiological extents of cell deformations, the permeability of the plasma membrane increases significantly, which is associated with the poration process. When a critical mass of cells have lost their homeostasis and then their viability through this deformation-inflicted damage pathway, microscopic tissue damage initiates and progresses. Following progression of local tissue damage and as en masse cell death occurs, the injury becomes macroscopic, including tissue breakdown that can be detected visually or by means of standard medical imaging such as ultrasound; however at this stage the damage is unlikely to be fully reversible.

Pressure injuries, when visible, can be in the form of blisters, non-blanching erythema, or areas of skin redness, or maroon purple or black spots on intact skin indicating subdermal tissue necrosis, or severe, open wounds after skin breakdown. The presence of tissue damage can also be manifested as changes to firmness (stiffness) of tissue detectable through palpation, or to localized tissue temperature detected by clinical inspection or through infrared thermography. Furthermore, indicators can be changed in the water contents of skin and subdermal tissues (indicating localized edema in the process of inflammation) which is detectable in an ultrasound examination or via a bioimpedance (sub-epidermal moisture) test. The areas that are predominantly prone to pressure injuries are areas that cover bony areas such as occiput, trochanters, sacrum, scapula bones, malleoli and the calcanei (heel) bones. The treatment of such conditions is excluded from the scope of the present invention as these constitute visible manifestations of late stages of PUs. The prevention of the development of such visible conditions is within the purposes of the present invention, as defined.

As known in the field, the definition of a pressure injury (as provided by the aforementioned NPUAP) may be divided into stages (e.g., for the purposes of treatment or prevention in accordance with the invention), namely:
Stage 1 pressure injury—Non-blanchable erythema of intact skin;
Stage 2 pressure injury—Partial-thickness skin loss with exposed dermis;
Stage 3 pressure injury—Full-thickness skin loss;
Stage 4 pressure injury—Full-thickness skin and tissue loss;
Unstageable pressure injury—Obscured full-thickness skin and tissue loss;
Deep pressure injury—Persistent non-blanchable deep red, maroon or purple discoloration.

Methods of the invention are directed at preventing the development of these stages.

In another aspect, the invention provides a method for preventing, arresting, slowing down, minimizing or modulating the onset and development of a pressure injury in a subject, by preventing and/or treating microscopic damages in a cell, group of cells, tissues, such as skin or any other tissue layer, or organ of said subject, the method comprising locally administering a formulation comprising an effective amount of a pyruvate compound or at least one pharmaceutically acceptable salt thereof to the skin region, or to a tissue, or to an organ of the subject suffering from or having predisposition to suffer from or susceptible to suffering from a pressure injury. As detailed above, the prevention, arrest, slowing down, minimization or modulation permitted by a method of the present invention generally refer to any of the following stages in the injury cascade: (1) first onset of cell damage, e.g., characterized by abnormal cytoskeletal changes and plasma membrane changes e.g. poration of the plasma membrane, (2) necrosis of individual cells as a result of the previous damage phase, (3) necrosis-apoptosis spiral resulting from the early necrotic cell death and leading to en masse cell death, (4) inflammatory response and localized edema triggered by the release of signaling molecules by dying cells at stages 2 & 3, (5) formation of a necrotic lump as a result of progress to macroscopic damage, and/or (6) appearance of visual signs of tissue damage on the skin (redness or other changes of color, texture, temperature or firmness) and/or skin breakdown and/or identification by standard medical imaging technologies. The invention is effective in influencing through any or all of the mid-points, and is as efficient if implemented early to prevent the early cell death and microscale damage.

The invention further provides a method for preventing, arresting, slowing down, minimizing or otherwise modulating the development of a pressure injury in a subject, the method comprising locally administering a formulation comprising an effective amount of a pyruvate compound or at least one pharmaceutically acceptable salt thereof to the skin region, to a subdermal tissue (e.g. through injection or transepidermal delivery), fat, muscle, tendon or any other connective tissue, or to an organ of the subject suffering from or having predisposition to suffer from or susceptible for suffering from a pressure injury.

In another aspect, the invention provides a method for treating/repairing microscopic damages in a cell, groups of cells, tissue, skin, subdermal tissue, fat, muscle, tendon or any other connective tissue or organ of a subject, said microscopic damage being caused by a pressure-inducing and/or shear-inducing force (i.e., a short term or long term pressure and shear to a skin region) to the cell, tissue, skin or any deeper tissue layer or organ, the method comprising locally administering a formulation comprising an effective amount of a pyruvate compound or at least one pharmaceutically acceptable salt thereof to the skin region or sub-dermally or to deep tissue layers, such as fat and muscle, or to an organ of the subject suffering from or having predisposition to suffer from or susceptible for suffering from a pressure injury.

Methods of the invention may be, in some embodiments, used to prevent pressure ulcers or pressure injuries that are not visible to a medical practitioner examining a subject visually or by standard medical imaging technologies. In some embodiments, methods of the invention may be preventing, arresting, slowing down, minimizing or otherwise modulating the development of a pressure injury in a subject who is susceptible for suffering from a pressure injury, but who has yet developed signs of such an injury.

Methods of the invention utilize a pyruvate, as defined, or a formulation or composition comprising same, that is “locally administered” to the subject. In other words, the pyruvate or a formulation or a composition comprising it may be administered by any form of administration intended to impart a local non-systemic effect. The local effect may be induced by administering the pyruvate or a formulation or a composition thereof to a body surface, such as a particular region of the skin in the body, or to any deep-skin tissue or organ, to thereby contain the tolerance enhancing effects of the administrated compound against damaging sustained deformations to cells and tissues at or near the suspected site, i.e., cutaneous, percutaneous, intracutaneous and/or intradermal, subdermal, or deep tissue structure including fat, muscle, tendon, connective tissues and the like region(s) and/or target(s), with little or no systemic absorption or accumulation of the compound or formulation.

In some embodiments, the subject being treated in accordance with the invention is a subject receiving additional treatment of pressure injuries or for any other disease or disorder (e.g. a diabetic patient receiving insulin, a cancer patient receiving chemotherapy or a malnourished patient receiving food supplements). For this reason and also for the purpose of achieving an effective treatment or prophylaxis, the pyruvate compound is provided in an effective amount and administered locally to the target site. The “effective amount” for purposes herein is determined by such considerations as may be known in the art. The amount must be effective to achieve a desired prophylaxis or treatment. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of biochemical/physiological response parameters such as half-life in the body, on factors such as age and gender, and others.

The “prevention or treatment” methods of the invention aim at providing means for treating and preventing manifestation of pressure ulcers before they occur, slow down progression of the condition, slow down deterioration of symptoms associated with early or progressive stages of the condition, enhance onset of recovery, slow down irreversible damage caused in progressive stages of the condition, delay onset of the progressive stages, lessen severity or cure the condition, improve survival rate or a more rapid recovery, or prevent the condition form occurring or a combination of two or more of the above.

The term “prevention” also encompasses treatment of microscopic damages caused to cells, which may deteriorate or develop into pressure ulcers or pressure injuries. Thus, in some embodiments, the term encompasses treatment of pressure injuries that are not visible upon examination of a subject, as further detailed herein.

The present invention further provides a method for preventing the development of pressure injuries, arresting pressure injuries at an early stage and/or reducing the recurrence of pressure injuries in a subject in risk of developing pressure injuries, the method comprising:
examining the subject's skin or tissues susceptible for injury for signs of microscopic damage;
determining or clinically judging whether said microscopic damage is indicative of an early stage pressure injury;
if early stage pressure injury is detected (or suspected), locally administering a formulation comprising an effective amount of a pyruvate compound, as defined herein, to the skin region or any other tissue type (e.g., deep tissues) or to the vicinity thereof where early stage pressure injury is detected or believed to be developing.

As used herein, the subject is a human being or an animal that suffers from a pressure injury or “a subject that is at risk of developing pressure injuries”. The subject at risk is a subject e.g., a surgical patient; a subject requiring investigation of a condition through medical imaging, e.g., MRI or vascular catheterization; a subject requiring a minimally invasive surgery such as endoscopy or arthroscopy; a female in labor administrated with epidural injection; a demented patient or an unconscious patient; bedridden patient either at home or hospitalized that following assessment by a medical professional (e.g., a person who is in charge or helps in identifying or preventing or treating an illness or a disability such as a physician, a tissue viability or a certified ward nurse, a nurse practitioner, a physician assistant, a midwife, a radiographer, a paramedic, a surgeon's assistant, a physiotherapist, an occupational therapist etc.) is considered to be at risk of developing a pressure injury based on various parameters, such as skin condition, incontinence, history of pressure injuries, consciousness, mental, physical and nutritional state of the subject, the subject's age, type of disease, background conditions and comorbidities, sedation, medications or anesthetic agents which have been prescribed, overall treatment objectives for the patient, repositioning frequency, level of activity and mobility or any other criteria that is used to determine the risk of developing pressure injuries known in the art. Additional criteria may also involve the hospitalization conditions such as friction, shear forces and interface pressure present in the bed/chair in which the subject spend most of his/her time, presence of moisture or wetness on the skin, etc.

Examination of a subject for detection of signs of skin damage is carried out (e.g., on admission to a hospital) as part of a risk assessment procedure upon acceptance to an institute/ward and routinely thereafter, according to practices and guidelines well-known in the art, such as the guidelines provided by NPUAP staging system or the Braden scale for risk assessment (Nurs. Res. 1987 July-August; 36(4):205-10) or similar commonly accepted scales such as Norton or Waterlow that are clinically used for routine risk assessment. In applying these tools, the medical practitioner, typically a nurse, detects and classifies pressure injuries (e.g., by searching for a break in the continuity of a skin surface, report of pain by a patient, presence of blanchable erythema or changes in pigmentation, sensation, temperature, or firmness of the skin, etc.).

As noted herein, examination of the subject's skin or tissues includes also the subdermal or deep tissues and is carried out, for example, first through visual skin inspection and then possibility through use of a dedicated technology or device, e.g., digital photography and subsequent image analysis of skin images, medical imaging (e.g., using portable ultrasound), infrared thermography, or bioimpedance (e.g., a sub-epidermal moisture test) or any other relevant available technology or combination thereof for early detection of the condition;

The pyruvate compound used in methods of the invention is pyruvic acid or an ester, amide or salt thereof.

In some embodiments, the pyruvate compound used is pyruvic acid.

In other embodiments, the pyruvate compound used is an ester of pyruvic acid. The pyruvic acid ester (pyruvate) may be selected from alkyl esters, and any substituted form thereof. In some embodiments, the alkyl ester is derived from alcohols having between 1 and 10 carbon atoms. Non-limiting examples include methyl pyruvate, ethyl pyruvate, propyl pyruvate, iso-propyl pyruvate, butyl pyruvate, t-butyl pyruvate, pentyl pyruvate, hexyl pyruvate, cyclohexyl pyruvate, heptyl pyruvate, octyl pyruvate, nonyl pyruvate, decyl pyruvate and others.

In some embodiments, the pyruvate ester is an ester of an alcohol comprising between 1 and 5 carbon atoms, e.g., methyl pyruvate, ethyl pyruvate, propyl pyruvate, iso-propyl pyruvate, butyl pyruvate, t-butyl pyruvate and pentyl pyruvate. In some embodiments, the pyruvate ester is an ester of an alcohol comprising between 1 and 3 carbon atoms.

In some embodiments, the pyruvate ester is methyl pyruvate, ethyl pyruvate or propyl pyruvate.

In other embodiments, the pyruvate compound is an amide of pyruvic acid. The amide is derived from an amine selected from ammonia, mono-alkyl amine, di-alkyl amine and tri-alkyl amine, wherein the alkyl is selected from C₁-C₁₀ alkyls. In some embodiments, the alkyl is selected from C₁-C₅ or C₁-C₃ alkyls.

In some embodiments, the pyruvate compound is a salt of pyruvic acid. The salt may be a base-addition salt derived from an organic or inorganic base, such as alkali metal hydroxides, including potassium hydroxide, sodium hydroxide, and lithium hydroxide; alkaline earth metal hydroxides, such as barium hydroxide and calcium hydroxide; alkali metal alkoxides, such as potassium ethanolate and sodium propanolate; and various organic bases such as piperidine, diethanolamine, and N-methylglutamine.

In some embodiments, the pyruvic acid salt is selected from sodium pyruvate, calcium pyruvate, zinc pyruvate, lithium pyruvate, potassium pyruvate, magnesium pyruvate, manganese pyruvate, and others.

In one embodiment, the pyruvate is sodium pyruvate.

The pyruvate compounds are used in a form that is pharmaceutically acceptable or has received regulatory approval (such as FDA) for non-pharmaceutical applications in human health, as food supplements or different kinds, skin creams and others.

Additional pharmaceutically acceptable salts that may be used in accordance with the invention may be found in, e.g., Remington's Pharmaceutical Sciences, 17th ed., Mack Publishing Co., Easton, Pa., 1985, p. 1418, which is incorporated herein by reference.

The pyruvate compound may be a single compound or a combination of two or more such compounds, not necessarily from the same chemical group. For example, where a combination of two or more pyruvate compounds is required, the two or more may be all salts, esters or amides or pyruvic acid, or a combination of a pyruvate salt and a pyruvate ester, or any other combination.

In some embodiments, a combination of two or more pyruvate compounds comprises pyruvic acid. In other embodiments, the combination is free of pyruvic acid.

In another aspect, the invention provides a pyruvate compound for use in a method of preventing or treating pressure injuries, as defined herein.

The invention further provides use of a pyruvate compound in the preparation of a composition for use in a method of treating or preventing a pressure injury.

Further provided is a use of a pyruvate compound in a method of treating or preventing a pressure injury.

The pyruvate compound may be formulated into a pharmaceutically acceptable formulation or into a form that is easily deliverable or administrable or into a form that may be used in the manufacture of a delivery device, e.g., a patch, and optionally in such a way that would allow the delivery or release of the compound from the device in a predetermined fashion, to allow slow, fast, immediate or suspended release. Thus, the invention further provides a formulation (or a composition) comprising at least one pyruvate compound for use in a method of treating or preventing pressure injury.

In some embodiments, the formulation is in a form adapted for a particular administration form. In some embodiments, the formulation may be formulated as an ointment, a foam, a gel, a lotion, a cream, a powder, a solution, a solution for injection, or formulated to be contained or carried or comprised in an article or a device, as further defined below, such as a dressing, a pad, a bandage, an underwear, a diaper, a plaster, a patch, a mattress, a cushion, a cover (for a seating surface or for a mattress), e.g., bedsheets, or any medical device which contacts the skin, e.g., CPAP mask, catheter, pulse oximeter, electrode etc., seating surfaces, e.g. toilet seats, and others.

In some embodiments, the formulation is adapted for injection. Injection may include different sites in the body and may be formulated for different depths at those sites, e.g. skin, muscle, fat and others.

In some embodiments, the formulation is adapted for transdermal delivery. In some embodiments, the formulation is adapted for topical or local administration.

In some embodiments, the formulation is adapted for manufacture of at least one article or device, as recited herein.

In accordance with the present invention, local administration may include any combination of the aforementioned means, e.g., a step of applying a dressing or a patch impregnated with a formulation of the invention to a pressure injury, or to a site where an injury may develop, securing the dressing to the subject's skin and co-administering an injection comprising a pyruvate formulation to the same or different regions of the skin or deeper tissues below the skin, as noted herein. Local administration may also be applied following therapeutic manipulation of a skin region or body tissues by systematic stroking, rubbing, kneading, massaging or application of short term pressure, to affect a physiological response that may be synergistic to the subsequent administration of the pyruvate, or which may assist in the pyruvate distribution in the region susceptible for developing pressure injury. In some embodiments, stretching of a skin region or a tissue is used, as further described herein.

The therapeutic manipulation, as above, e.g., stretching, may be applied after local administration of the pyruvate, prior to administration, or at any stage during a pyruvate administration protocol.

Formulations according to the invention may comprise, in addition to the pyruvate compound(s), a variety of non-active or active additives. The additives may be selected to dissolve or carry the pyruvate compound(s) and aid local delivery and improve bioavailability (e.g., by improving skin penetration and/or permeability).

Formulations of the invention comprise pharmaceutically acceptable carriers such as vehicles, adjuvants, excipients, diluents, cell-fusogenic liposomal-carriers or nano-carriers that are chemically inert to the pyruvate compound(s) and have no detrimental side effects or toxicity under the conditions of use.

In some embodiments, the formulations of the invention may be administered using drug delivery methods, for example, selected from ultrasound stimulation, electrical stimulation or heat stimulation (e.g., to aid in the penetration of the administrated compound into skin and deep tissues).

The choice of carrier or delivery method will be determined in part by the particular form of the pyruvate compound, as well as by the particular method used to administer the compound or composition comprising it or by the tissue to which the compound is targeted. Accordingly, there is a wide variety of suitable formulations of the pharmaceutical composition of the invention.

Generally speaking, depending on the method of administration, the formulation may be formed as a liquid formulation or as a solid formulation. Liquid formulations may comprise an effective amount of the pyruvate compound dissolved in a diluent, such as water, saline, or any other liquid-phase material. Solid formulations may be formed as solids, granules, powders suitable for solid-phase delivery or as solutions or suspensions or emulsions or creams in an appropriate liquid.

Liquid formulations may include diluents, such as water and alcohols, for example, ethanol, benzyl alcohol and polyethylene alcohols, either with or without the addition of a pharmaceutically acceptable surfactant, suspending agents, emulsifying agents, lubricants, inert fillers, buffering agents, stabilizing agents, disintegrating agents, moistening agents, preservatives, and biocompatible carriers.

The pyruvate compounds may be made into injectable formulations. The requirements for effective biocompatible-carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Formulations of the invention may be locally injected to a patient prior to undergoing a medical procedure (e.g. surgical or imaging examination or endoscopic investigation) that requires a period of more than 10 minutes of immobilization and/or impaired sensory response or sensitivity or involves hospitalization period where reduced or lack of a subject's motility is expected, or any procedure that in course thereof pressure is applied to a skin region of the subject. For example, at hospitalization, prior to surgery, the subject who is assessed to be at risk of developing pressure ulcers (e.g. a diabetic patient) is carefully inspected for any skin micro-damage or redness, especially over bony areas. Based on clinical judgement of risk of pressure injury to a certain anatomical site, or upon detection of each of the aforementioned damage indicators and determination of an early stage pressure injury or without indications at prone sites—the composition of the invention is locally injected or otherwise administrated into a region(s) prone to develop a pressure ulcer (e.g., into soft tissues at body sites under supported bony prominences at several or specific tissue depths at the skin-surface or below at deeper tissues as determined through the potential risk, or at a region subjected to mechanical loads by a medical device) before the patient is positioned in the bed or chair. Thus, the application can be at sites where preliminary indications of pressure injury have been detected or at prone sites where pressure injuries area likely to form yet without indications as a treatment or a preventative.

In some embodiments, the formulation of the invention is administered by means providing slow release or controlled release of the pyruvate or a formulation comprising same, e.g., through transepidermal delivery of a composition of the invention. This provides a tool that covers both prophylaxis application and treatment of potential early damage, which essentially prevents the formation or inhibits the progression of a very early stage pressure injury into progressive, e.g., unstageable or subdermal or deep pressure injury and enhances tolerance and resistance of cells within the tissues to sustained deformations.

The formulation may be administered at pre-defined time intervals (e.g., once every 15 minutes or once per hour or once per day or once per week) or may be administered 1, 2, 3, 4, 5 or up to 10 times, depending on the physical condition of the patient, the level of risk at which they are determined to be, the development and condition of the subject's pressure injuries or on any other parameter which the medical professional considers relevant to require additional recurrent administration of the formulation of the invention.

In some embodiments, the formulation of the invention is administered concomitantly with general care protocols, with routine skin assessment protocols, with repositioning protocols when applicable, with at least one additional medicament or with use of any number of non-medicinal preventative measures, e.g. support surfaces.

In some embodiments, the formulation of the invention is administered sequentially prior to or after administration of the at least one additional medicament.

In accordance with the present invention, the concomitant or sequential administration of a formulation of the invention in combination with the at least one additional medicament may be continuous over a time period (e.g., of fractions of an hour, or hours, days, weeks or month) until the risk has ended (e.g., anesthesia has cleared) or until recovery of the subject from the immobilizing condition.

In some embodiments, at least one additional medicament may be administered systemically, topically or by any other suitable route of administration, independent from the formulation of the invention which is locally administered as defined herein.

In other embodiments, at least one non-medicinal preventative measure may be applied independently from the formulation of the invention.

Notably, the herein disclosed methods for preventing the development of pressure injuries or arresting pressure injuries at an early stage are translatable into the clinic and have the potential to ultimately transform hospital care, home care, nursing facility care, community care and others by preventing various medical complications.

As noted herein, formulations of the invention may be in a form of a liquid, semi-solid or solid composition for application directly to the surface of the site at-risk of pressure injury or the suspected or prone pressure injury site (e.g., applying a cream or ointment directly onto the pressure injury) or for indirect application to an early stage pressure injury (e.g., incorporated into a solid contacting layer such as a dressing).

In some embodiments, formulations may be incorporated into a dressing or a patch. The dressing or patch may be any suitable dressing or patch that is commonly used in the art to prevent pressure injury (commonly referred to in the art as prophylactic dressings) or dressings used to treat wounds and/or pressure injuries. In some embodiments, the dressing is selected from a gauze, an impregnated gauze (e.g., gauze dressings impregnated with additional substances such as petroleum, iodine, bismuth, and zinc), a transparent or nontransparent film dressing, a foam dressing, a hydrogel, a hydrocolloid dressing, an alginate, a hydrofiber dressing, a silicone dressing and a silver dressing.

The dressing may be maintained in contact with the intact (normal) skin or the early lesion or at the prone or suspected site of pressure injury for varying time periods depending on various parameters associated with the risk of the individual, condition of the early stage pressure injury, if exists, and the condition of the subject as readily recognized by the person versed in the art.

In some embodiments, the dressing is maintained in contact with the skin for a period of between 10 minutes and 5 days, or for at least 23 of 24 hours of every day where the subject is bed-ridden.

In some embodiments, the dressing is configured to release to the skin an effective amount, as defined herein, or up to about 100 mM of a pyruvate compound for the intended period of dressing placement on the skin.

In some embodiments, the dressing or patch or foam comprises a matrix (e.g., alginate, collagen, or a synthetic bioabsorbable polymer) to allow the slow release of a pyruvate compound from the dressing/patch/foam/polymer into the skin of the subject treated with the formulation of the invention.

In some embodiments, the matrix is embedded in a medical device e.g., CPAP mask or a coating of an endotracheal or a nasogastric tube to allow slow release from the medical device to the contacting skin.

In some embodiments, the formulation of the invention is administered by a combination of topical administration (e.g., cream, dressing, patch) and injection. In accordance with such embodiments, a formulation may be applied topically at one or more sites (e.g., the periphery and/or center) of the skin and an injection may be administered concomitantly or sequentially to the same region or to a different region of the skin or to deeper tissues. The number of injections and the amount of administrated pyruvate compound will be varied in accordance with various parameters (e.g., the size of the site that is prone to pressure injury, the condition of the patient, etc.).

As described herein, the formulation administrated topically (e.g., by dressing) may be administered prior to or together with a formulation administered by injection. In cases where concomitant topical and administration by injection are employed, the topical administration may continue as long as the treatment by injection is ongoing or to any period of time after the treatment by injection has been discontinued, e.g., 15 minutes to 30 days during the ongoing treatment by injection, or 15 minutes to 30 days after the treatment by injection has been discontinued. In cases where the topical administration of the formulation is begun prior to treatment by injection, topical treatment may begin, e.g., 15 minutes to 30 days before treatment by injection begins.

In some embodiments, the formulation of the invention is for use in preventing the development of pressure injuries, arresting pressure injuries at an early stage and/or reducing the recurrence of pressure injuries in a patient at risk of developing pressure injuries. In accordance with such embodiments, the amount of a pyruvate compound and/or any other additional material included in the formulation may be the same or different than in a formulation used for treating existing early stage pressure injuries.

The dosage regimen of the pyruvate compounds and formulations of the invention may vary depending upon known factors such as the particular material (e.g., type of pyruvate salt), the patient's age, sex, health, medical condition, risk level, body habitus and weight; the nature and extent of the symptoms to be expected and prevented; the kind of concurrent treatment; the instrumentation used in concurrent treatments; the frequency of treatment with the compounds and formulation; the expected time of immobilization or sensory impairment. The pyruvate compound(s) utilized in accordance with the present invention may be administered in a single daily dose, e.g., to be released continuously through a controlled release or slow release device, or the total daily dosage may be administered in divided doses of two, three, four or more times daily.

In still another of its aspects, the present invention provides a kit for use in the treatment of early stage pressure injuries (as means to prevent the development of such injuries), the kit comprising instructions of use and:

a) a formulation comprising an effective amount of a pyruvate compound or a composition thereof; and optionally

b) means for applying the formulation to a skin region or deeper tissues of a subject.

In some embodiments, the means for applying the formulation to the skin of a subject is selected from a dressing, a pad (e.g., gauze pad), a bandage, an underwear, a diaper, a plaster, a patch, a syringe-injection, a mattress or a cushion, a cover or bedsheets, or a medical device in contact with the skin (e.g., CPAP masks, catheters, pulse oximeter, tubing, electrodes and wiring).

The present invention further provides a method for preventing and/or treating a pressure ulcer (formation and development) in a subject, the method comprising locally administering a formulation comprising an effective amount of a pyruvate compound or at least one pharmaceutically acceptable salt thereof, to a skin region or a deeper tissue region(s) or to an organ of the subject who is at risk of or showing signs of a pressure ulcer, wherein a low- or medium-level stretching strain is applied to said skin region or to said deeper tissue region(s) or to said organ of the subject prior to or concomitant with the administration of said formulation.

In another of its aspects, the invention provides a method for preventing, arresting, slowing down, minimizing or modulating the onset or the development of a pressure injury in a subject, by preventing and/or treating or repairing microscopic damages in a cell, group of cells, tissue such as skin or any other tissue layer, including subdermal or deep tissues, or an organ of said subject, the method comprises locally administering a formulation comprising an effective amount of a pyruvate compound or at least one pharmaceutically acceptable salt thereof, to a skin region, to a subdermal tissue, to a deep tissue (e.g., muscle) or to an organ of the subject suffering from or having predisposition to suffer from or susceptible to suffering from a pressure injury, wherein a low- or medium-level stretching strain is applied to said skin region or to deep tissue at site or to the organ of the subject prior to or concomitant with the administration of said formulation or following its removal.

In still another one of its aspects the invention provides a method for preventing, arresting, slowing down, minimizing or modulating the onset and the development of a pressure injury in a subject, the method comprising locally administering a formulation comprising an effective amount of a pyruvate compound or at least one pharmaceutically acceptable salt thereof to a skin region, to a subdermal tissue, fat, muscle, tendon or any connective tissue, or to an organ of the subject having a predisposition to suffer from or susceptible to suffering from a pressure injury, wherein low- or medium-level stretching forces are applied to said skin region, a subdermal tissue, fat, muscle, tendon or any connective tissue, or to an organ of the subject prior to or concomitant with the administration of said formulation or following its removal.

In still yet another one of its aspects the invention provides a method for preventing and/or repairing microscopic damages in a cell, groups of cells, tissue, skin, subdermal tissue, fat, muscle, tendon or any other connective tissue or organ of a subject, said microscopic damage caused by pressure-inducing and/or shear-inducing forces to the cell, groups of cells, tissue, skin or a deeper tissue layer, or organ, the method comprising locally administering a formulation comprising an effective amount of a pyruvate compound or at least one pharmaceutically acceptable salt thereof to a skin region or sub-dermally or to a deep tissue layer of the same or different type or to an organ of the subject having predisposition to suffer from or susceptible to suffering from a pressure injury, wherein a low- or medium-level stretching strain is applied to said skin region or sub-dermal or a deep tissue layer or organ of the subject prior to or concomitant with the administration of said formulation or following its removal.

The invention also provides a method for preventing onset of a pressure injury, repairing microdamage, arresting development of a pressure injury at an early stage and/or reducing the recurrence of a pressure injury in a subject in risk of developing pressure injuries, the method comprising:
determining that the subject is at risk for a pressure injury, based on clinical judgement or a medical examination or a risk assessment that is clinical, or is based on measurements by means of a device, or is a combination thereof;
or, examining the subject's skin or other tissues susceptible for injury for signs of microscopic or early cell/tissue damage, visually and/or by means of a medical device that provides indication for damage, e.g., for example (and without loss of generality) based on ultrasound, bio-impedance (e.g. changes in resistance and/or capacitance and/or dielectric properties of cells/tissues), infrared spectroscopy or near-infrared emission or thermal measurements;
or, determining or clinically judging whether said microscopic damage is indicative of early stage pressure injury;

if a risk for a pressure injury is determined and/or if early stage pressure injury is suspected or detected, locally administering a formulation comprising an effective amount of a pyruvate compound to a skin region or any tissue or vicinity thereof where early stage pressure injury is suspected/detected, and applying a low- or medium-level stretching force/deformation/strain which could be either sustained or dynamic (e.g., cyclic), to said skin region or any other tissue type or the vicinity thereof the subject prior to or concomitant with the administration of said formulation or following its removal.

Also provided by the present invention is a kit for the treatment of early stage pressure injuries, the kit comprising instructions of use and:

a) a formulation comprising an effective amount of a pyruvate compound or a composition thereof;

b) means for applying the formulation to the skin or deeper tissues of a subject;

c) means for applying a low- or medium-level stretching force/deformation/strain to the skin or deeper tissues of a subject in association with the administration of the said formulation.

As used herein, the term “low- or medium-level stretching force or deformation or strain” generally refers to a tensional mechanical load that is applied to a skin region or to deeper tissues in a non-invasive manner The stretching may be applied using any suitable layer or article or device or consumable that is attached to or generally brought into contact with any skin region or PU (e.g., margins of the PU) or region/s prone to develop a PU. The layer, article or device may be in any form suitable also for applying the formulation to the skin region of a subject, as described herein; thus the layer, article or device may be in a form of e.g., a dressing, a pad, a gauze pad, a bandage, an underwear, a diaper, a plaster, a patch, a gel or a medical device or consumable in contact with the skin region. To maintain intimate contact with the skin region, the layer, article or device or consumable may have an adhesive layer on at least a region of its surface, to thereby securely and tightly attach it to the skin region. A force/deformation/strain may be applied mechanically, thermally, magnetically, electrically, thermo-mechanically, magneto-mechanically, via a chemical response between two or more components or in any other combination therein, and may be powered or non-powered. A non-limiting example is schematically presented in FIG. 1C, enabling application of controlled, sustained, radial stretching to the skin tissues and deeper tissues near a skin region through direct contact with the skin region.

An exemplary device such as that exemplified in FIG. 1C comprises (1) a ring-like, skin-adhesive, stretchable (e.g. elastic) material; (2) a stretching element constructed or engineered for applying stretching for example but not limited to mechanical, magnetic, magneto-mechanical or electrical means or via a chemical reaction, that may be applied manually or automatically or semi-automatically or otherwise initiated and activated by physical contact of an operator with the layer/article/device/consumable or remotely (e.g. via a controlling computer or device, tablet, cellular phone or the like); and (3) a ring-like, skin-adhesive, non-stretchable (e.g. stiff and elastic) material that serves as a physical anchor to define the maximal applicable stretching to prevent application of stretching to potential damage-levels. The stretching element may be a single elastic strip placed between the rings as indicated, or any other configuration that may be continuous or non-continuous, circular/annular or of any other shape or geometry.

As shown in FIG. 1C, the device may be constructed of two concentric rings. The inner ring is of a skin-adhesive, stretchable (e.g. elastic) material selected of materials responsive to e.g., micro-environmental conditions, such as exposure to e.g. air, skin, body-heat, sweat or any other conditions typical to treated body regions. Alternatively, the aforementioned material may be responsive to electrical current or voltage, external heating or cooling, piezoelectricity, thermo-responsiveness, chemical reactions generating stress or force, electromagnetic or magneto-mechanical stimulation. The inner ring of the device may be stretched, in some embodiments, by mechanical means, for example by one or more pulling strips or elastic strips positioned between the rings, or by any other configuration that is continuous or non-continuous, circular/annular or of any other shape or geometry. The outer ring of the device, for example in the ring structure, may be composed of a skin-adhesive, non-stretchable (e.g. stiff and elastic) material that serves as a physical anchor to define the maximal applicable stretching to prevent application of stretching to damage-potential levels.

In some embodiments, the material of the inner ring may be responsive to skin-environmental conditions and may change shape and induce stretching following exposure to air, skin, body-heat, sweat (perspiration), pH changes or any other condition typical of treated areas and skin regions in a subject.

In some embodiments, the device is designed for separate use or to be combined e.g. with prophylactic dressings, bandages, patches, garments, underwear, gels or any other element in contact with the skin.

In other embodiments, the device is used in combination with administration of at least one pyruvate compound, as defined herein. In some embodiments, the compound is contained within a device element or material or consumable and is released therefrom upon contact with the skin region.

In some embodiments, the device may be associated with or used in conjunction with at least one further layer, article or device or consumable selected, in a non-limiting fashion from prophylactic dressings, patches, garments, underwear, gels or any equivalent layer, article or device or consumable that is brought into contact with the skin region.

The layer, article or device or consumable may be attached to the skin region to apply the herein described low- or medium-level stretching strain using a non-invasive (e.g., medical adhesive) measure or manipulation, depending on the condition and location of the PU/skin region/tissue prone to develop a PU and the judgment of the medical professional.

The low- or medium-level stretching strain, as described herein, is applied relative to the skin at rest. The stretching strain is a low to medium level strain that stretches the skin in one direction or in multiple directions. Stretching may also be radially directed. According to the invention, a low to medium level of stretching generally refers to an elongation in the skin region from a resting position to a stretched position and is typically measured in percent strain (elongation). The stretching causes a strain in levels below cell- or tissue-damage inflicting strains. In some embodiments, tissue stretching is held constant and is always lower than the skin failure strain. In some embodiments, the strain levels that irreversibly damage a cell within a skin region or any deeper tissue is at a level of 12%. In some embodiments, the stretching is smaller than 10%, 8%, 6%, or 4%. In some embodiments, stretching is between 3 and 12%, or between 3 and 10% or between 3 and 8% or between 3 and 6%.

The level of stretching to be applied to the skin region or organ of the subject depends on various parameters, such as the amount pyruvate compound in the formulation, time of application relative to pyruvate compound treatment, the body part to be treated, the level of risk determined clinically, by a risk assessment process or through measurements with a designated device, or based on the shape, size of and severity of the suspected or existing PU, the general medical condition of the patient, etc.

The adhesive layer used for applying the herein described stretching force may extend over the whole surface region of the PU or region prone to develop a PU, may cover only certain parts of said surface and may also cover (e.g., unaffected/undamaged) region of skin adjacent to the PU or region prone to develop a PU.

In some embodiments, the stretching is applied to said skin region, tissue or organ of the subject prior the administration of said formulation.

In other embodiments, the stretching is applied to said skin region, tissue or organ of the subject concomitant with the administration of said formulation.

In other embodiments, the stretching is applied to said skin region, tissue or organ of the subject following removal of said formulation (or after discontinuation of treatment with a pyruvate compound).

Thus, according to a non-limiting embodiment of the present invention, a medical adhesive, patch or dressing is used to secure a dressing to the skin in the lower backside of a patient to apply a stretching strain below damage inducing levels, for example below 12% strain, to the skin region suspected with onset of pressure injuries or being affected with microscopic damage. After a period of 10 minutes and until such a time when the medical adhesive, patch or dressing are changed (e.g., after 2 days, 3 days, 5 days or a week), the patient may be administered with a formulation of the invention (e.g., by injection into the margins of the said affected area).

The invention further provides a device or an article comprising a releasable pyruvate compound for use in the prevention of pressure ulcers, the device or article being selected from a dressing, a pad, a bandage, an underwear, a diaper, a plaster, a patch, a mattress, a cushion, a seating surface, a surgical table, an examination table, a continuous positive airway pressure mask, an oxygen mask, a spinal board, a cervical collar, a pulse oximeter, a catheter, wiring, an electrode, a tracheostomy device, a nasogastric tube, an endotracheal tube, compression stockings, a cast, a positioner, a heel boot, a headrest, a footrest, a lying surface and a cover.

In some embodiments, the device of the invention is a device according to FIG. 1C. The invention further provides a device for inducing deformation of a skin region of a subject, the device comprising:

an outer, non-deformable or minimally-deformable frame element, having at least one first skin-contacting surface configured to adhere to a first skin region of the subject;

at least one inner elastically, visco-elastically or plastically deformable element, having at least one second skin-contacting surface that is configured to adhere to a second skin region of the subject; wherein the second skin region is within boundaries formed by the outer frame element; and

a member disposed between and connecting said outer element and inner element; wherein the member is elastic, viscoelastic or plastic;

wherein the elastic or viscoelastic or plastic member is configured to be selectively deformable, thereby causing elastic or viscoelastic or plastic deformation of the at least one inner element and said second skin region.

The outer frame element and the inner frame element may be of any shape. In some embodiments, one or both of the outer frame and inner frame is in the form of a ring, an ellipse or a polygon or a closed curved (concave or convex) path or non-closed path or form or any combination thereof.

The frames may be concentric or non-concentric relative to each other. Typically but not necessarily, the inner frame is concentric with the outer frame such that the inner frame is in contact with a skin region that is contained within boundaries formed by the outer ring. In other words, in this embodiment, the first skin region is concentric with the second skin region. Deformation or stretching of the inner ring, as disclosed, deforms the second skin region within the boundaries formed by the outer ring.

The elastic, viscoelastic or plastic member is configured to selectively deform upon application of any one of a mechanical force, a magnetic force, a magneto-mechanical force, an electrical input, a chemical reaction, a temperature change and at least one skin-environmental condition. In some embodiments, the elastic, viscoelastic or plastic member is induced to deform in response to at least one skin-environmental condition selected from exposure to air, skin, body-heat, perspiration, pH changes, or any combination thereof.

Alternatively, the selective deformation of the elastic, viscoelastic or plastic member may be user-induced, e g , manually, automatically, responsively or semi-automatically. In some embodiments, the inner element may be configured to be deformed by user-applied mechanical force, e.g., where the inner element is configured with a pulling handle or strip or thread or cord or fiber/s or fabric or a combination of which to permit a user to apply mechanical force onto the inner element.

The device may be designed for separate use or to be combined with prophylactic dressings, bandages, patches, garments, underwear, gels or for use in combination with any other device. As noted herein, a stretching device may be combined with administration of at least one pyruvate compound. The compound may be administered separately from the application of stretching, or may be contained within the device inner or outer element material, or generally contained at any skin-contacting element or region of the device, such that during application or while the device surface is in contact with the skin region, the at least one pyruvate is administrated to the skin.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-C depict various stretching devices according to the invention. FIGS. 1A and 1B depict a three-dimensionally printed cell stretching device (complete view FIG. 1B) for application of variable radial stretching levels to in vitro cells in culture. As shown in FIG. 1A, the device consists of a bottom plate on which 6 vertical cylinders are placed. A stretchable-substrata plate with 6 sample wells is placed on the bottom plate and the cylinders and under a top plate that is moved downwards to induce stretching of the 6-well plate substrata on the cylinders; distance between the top and bottom plates is controlled by screws and top plate is strengthened by flat plates at the screw locations. FIG. 1C depicts a schematic representation of a prototype device for application of low-level stretching to the in vivo skin regions and deeper tissues through the skin. The exemplary device includes (1) a ring-like, skin-adhesive, stretchable (e.g. elastic) material; (2) stretching element, applying stretching by mechanical, magnetic, magneto-mechanical or electrical means. This element may be a single elastic strip placed between the rings as indicated, or any other configuration that is continuous or non-continuous, circular/annular or any other shape; (3) a ring-like, skin-adhesive, non-stretchable (e.g. stiff and elastic) material that will serve as physical anchor to define the maximal applicable stretching to prevent application of stretching to damage-potential levels.

FIG. 2 shows an experimental protocol for evaluating cell membrane permeability following damaging tensile-strain. Cells were cultured on elastic substrates for 4 days in growth media (with 0 or 1 mM Sodium pyruvate). In the experiment (starting t=0), cells were pretreated with different sodium pyruvate concentrations (0, 1 and 5 mM) for 4 hours and then sustained 12% strain (or 0% control) was applied for 3 hours. In the last 30 min of stretching 10⁻⁴ M FITC-labeled 4 kDa dextran were added to half of the wells (unstained cells provide autoflourescence blank). Cells were then collected and dextran uptake was measured using flow cytometry relative to unstretched control. Viability was determined by calcein staining, by flow cytometry gating and by cell counting.

FIGS. 3A-C provide fluorescent distribution curves obtained from FACS system for C2C12 myoblasts cultured in 0 mM NaPy (NaPy free) growth media prior to an experiment; similar plots were obtained for the NIH3T3. The results demonstrate the effect on membrane permeability for pre-treatment with (FIG. 3A) 0 mM, (FIG. 3B) 1 mM, and (FIG. 3C) 5 mM NaPy. The two curves on the left of each panel are strained and unstrained controls (without fluorescent dextran, dashed line shows that those are indistinguishable). The two curves on the right of each panel correspond to unstrained (left) and strained (right) conditions, where reducing distance between then as NaPy pre-treatment concentration is increased shows reduction in strain-damage induced membrane poration.

FIGS. 4A and 4B show an increase in fluorescence intensity due to uptake of FITC-labeled 4 kDa Dextran into (FIG. 4A) C2C12 myoblasts (FIG. 4B) NIH-3T3 fibroblasts, both pre-treated with Sodium pyruvate (0, 1 and 5 mM) for 4 hours prior to exposure to 12% sustained stretch for 3 hrs. Cells were grown in growth media (GM) without Sodium pyruvate (light) or with 1 mM Sodium pyruvate (dark). Error bars are standard errors. Statistical significance was tested, where *p<0.03, ** p<0.0002.

FIGS. 5A-D depict kinematics of gap closure of C2C12 myoblasts (left, A and C) and NIH3T3 fibroblasts (right, B and D) for varying stretching levels and sodium pyruvate media-supplement. (FIGS. 5A-B) Normalized maximum migration rate; (FIGS. 5C-D) Time for closure of 90% of initial gap area. Asterisks mark statistically significant results (p<0.05) as compared to values with the same stretching level or with the same sodium pyruvate concentration, in the same cell type.

FIGS. 6A and 6B depict a three-dimensional (3D) finite element model of the buttocks. Axial cross-section (FIG. 6A) of the mesh with an enlarged tilted view (FIG. 6B) shows the elements distribution at the skin and fat tissue layers closest to the mattress.

FIGS. 7A-7C depict the damage score threshold 3D plots for (FIG. 7A) Epidermis, (FIG. 7B) Fat and (FIG. 7C) Muscle tissue, caused by applied pressure and temperature for varying durations, reconstructed using the equations developed by Kokate et al. (Wounds 9(4): 111-121 (1997). The plotted surface represents a unity damage score, a value which was determined by the original authors as the damage threshold.

FIGS. 8A-D demonstrate the development of a computational modeling framework used to conduct studies simulating release of sodium pyruvate (NaPy) from a novel prophylactic dressing: (FIG. 8A) Optical coherence tomography (OCT) image of the sacral skin of a 67 years-old female which has been used for the OCT-based modeling. (FIG. 8B) The four anatomical model variants, representing four levels of skin roughness (R1-R4). (FIG. 8C) Zoom-in on the stratum corneum (SC) and epidermis layers of the skin. The roughness criterion was set as the vertical distance between the top of the highest fold to the bottom of the lowest valley. (FIG. 8D) The thin-slice transient finite element computational model of NaPy diffusing from the dressing (set as an infinite reservoir here) into the skin.

FIG. 9 is an example of a time course of a change of concentration of sodium pyruvate (NaPy) molecules entering into the depth of the skin (in model variant R2).

FIGS. 10A-D provide average sodium pyruvate (NaPy) concentrations recorded during 16 hours of simulated use of the novel NaPy-releasing dressing, in each of the model variants representing four levels of skin roughness (R1-R4), in (FIG. 10A) the stratum corneum and epidermis layers, 1 mm (FIG. 10B), 2 mm (FIG. 10C), and 3 mm (FIG. 10D) deep into the dermis.

FIG. 11 shows time from dressing application to sodium pyruvate (NaPy) concentrations reaching a steady-state, for the four skin roughness levels (R1-R4), in the stratum corneum and epidermis, and 1, 2, or 3 mm deep into the dermis. Steady-state has been defined to occur when NaPy concentration was within ±10% of the plateau NaPy concentration value.

### DETAILED EMBODIMENTS OF THE INVENTION

To evaluate the efficacy of sodium pyruvate (NaPy) as a cytoprotectant material for pressure ulcer prevention, two cell functions were evaluated: (a) changes in the plasma membrane permeability of cells following damage caused by static, damage-level (12%) stretching; those indicate initial damage to cells leading to death, and (b) effects of sodium pyruvate in combination with low-level stretching (3-6%) on the rate of closure of microscale gaps; those were evaluated in the context of prevention. Closure of microscale gaps can prevent the enlargement of gaps into wounds. Both damage-level and low-level stretching were applied using a custom-made radial cell stretching device. Cell experiments were performed on NIH-3T3 fibroblasts (connective tissue and skin cells) and C2C12 myoblasts (muscle precursor cells). The C2C12 are undifferentiated muscle cells, which are the migratory fraction of cells that can migrate to close any micro-damage. A cell-damaging load of 12% stretching was selected based on Slomka N and Gefen A (Ann. Biomed. Eng. 40(3), 606, 2012) and was applied using a custom radial cell-stretching device (Toume, et al., J. Biomech. 49: 1336 (2016)).

### Cell Culture Protocols

Cell culture: undifferentiated C2C12 myoblasts (CRL1722, ATCC, VA) or NIH3T3 cells (CRL-1658) were cultured (separately) in a growth media containing Dulbecco's Modified Eagle's medium (with 4.5 g/L glucose,) 4mM L-glutamine, 1% and Penicillin G+Streptomycin, and 10% or 20% fetal bovine serum (FBS) was respectively added to the C2C12 and the NIH3T3 (all from Biological Industries, Beit-Haemek, Israel). In addition, effects of sodium pyruvate were evaluated in growth media supplement on both cell types: adding 1 mM NaPy as typical media supplement to enhance cell proliferation or growing cells in NaPy free media as expected in physiological conditions in the body; NaPy levels in human blood serum are 0.090-0.12 mM (O'Donnell-Tormey, et al., J. Exp. Med. 165: 500-514 (1987)). Thus, damage-testing was performed on cells that had been cultured in growth media that is supplemented with 0/1 mM NaPy for several days prior to the experiment, respectively, for physiological relevance and for typical cell culture conditions. Cells were incubated at 37° C., and 5% CO₂ and were passaged when confluent (˜80%) every 3-4 days. The cells used for all experiments were always at passages under 30.

### Cell Stretching Device

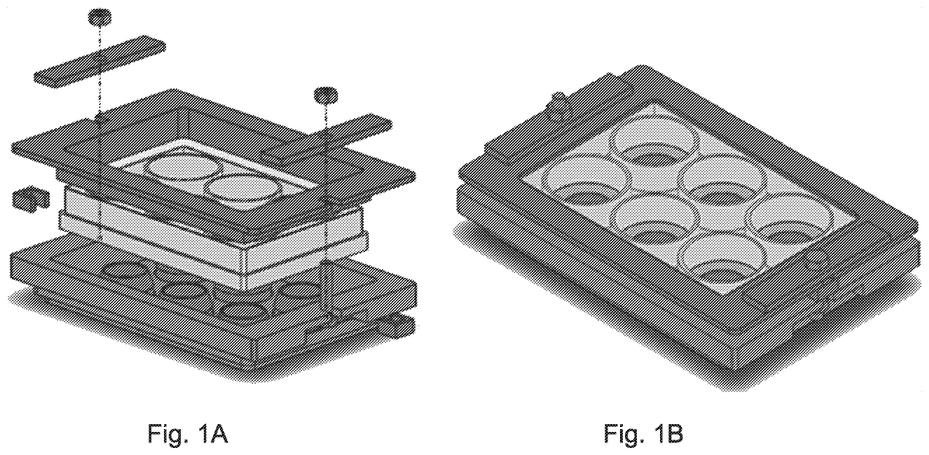

Radial stretching was applied to an elastic substratum on which cells are cultured. Cells were cultured on a bioflexcell collagen-coated 6-well plate (Flexcell Inc., North Carolina) that was placed between two rigid detachable frames (20×11×6 cm³) and on top of 6 centrally located vertical cylinders (FIG. 1). The top frame, which was also a cover to the sterile bioflexcell plate, was pushed downwards towards the bottom frame, inducing contact between the elastic substrata and the vertical cylinders. When pushed down the elastic substrata was stretched radially, and the strain was determined by the distance between the top and bottom plates. To maintain a static (constant) strain in the substrata a set of rigid spacers were used to define the distance and screws to reach and maintain it.

Sodium Pyruvate Pre-Treatment Reduces Cell-Membrane Permeability and Cell Death Resulting from Damage-Level Strains

### Membrane Permeability Experimental Protocol

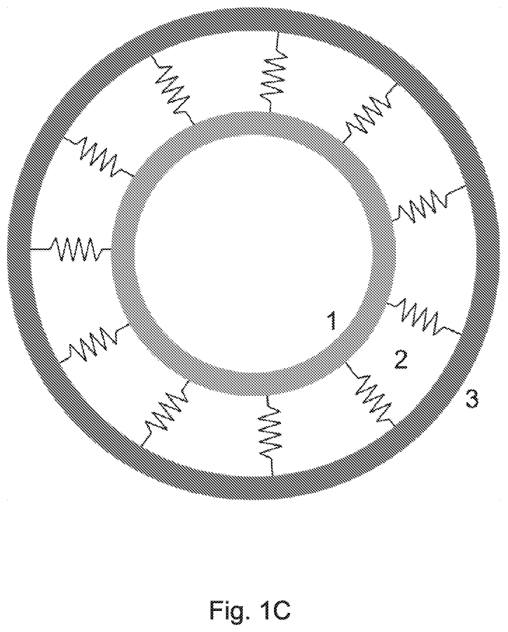

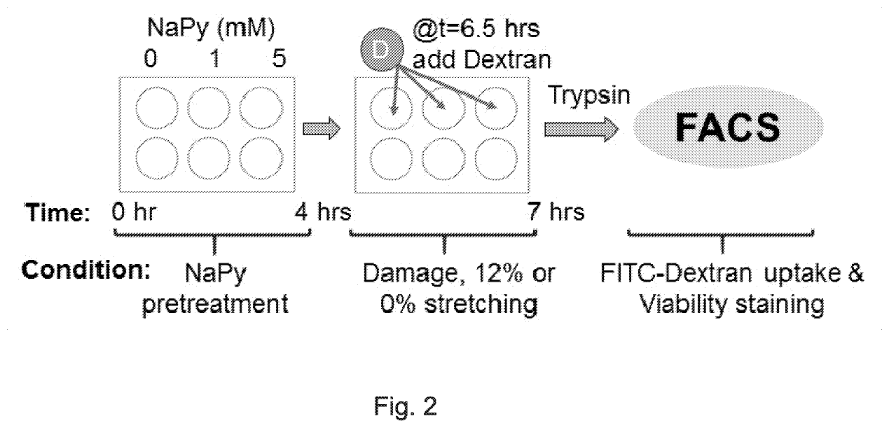

For each experiment, 300,000 cells were cultured 24 hours in parallel on 2-4 bioflexcell collagen-coated 6-well plates (Flexcell Inc., North Carolina). On the day of the experiment the cell growth media (with 0/1 mM NaPy) was replaced with media supplemented with different NaPy concentrations (0, 1 or 5 mM) and cells were incubated for 4 hours; this constitutes the pre-treatment of the cells. A four-hour pre-treatment was chosen as that is a clinically relevant time-scale during preparation e.g. before planned surgical procedures. A flow diagram of the experimental protocol is provided in FIG. 2.

Each 6-well plate contained two wells with each of the three NaPy pre-treatment concentrations. After 4 hours, all but one of the bioflexcell plates were simultaneously subjected to damaging-level, sustained strain-loads of 12% radial stretching for 3 hours and one plate in each experiment was not stretched for control. After 2.5 hours of stretching, 0.1 mM of fluorescein isothiocyanate (FITC)-labeled 4 kDa Dextran (Sigma-Aldrich, Israel) was added to one of the wells of each NaPy pre-treatment concentrations for the remaining stretch-loading duration of 30 min; the low molecular weight Dextran molecule can pass through small membrane-pores providing a sensitive measure for initial poration. Following the incubation period, the substrate stretching was relieved and cells in all wells were kept in their media for an additional 10. Cells were then rinsed twice with PBS to remove any non-internalized fluorescent stain and cell debris. Then adhered cells were detached with trypsin and suspended in serum-containing growth media to inhibit trypsin activity. Next, cells were centrifuged (900 rpm for 5 min) and resuspended in 0.5 ml of PBS within 12×75 mm polystyrene test tubes for use with fluorescence activated cell sorting (FACS).

Permeability of the plasma membrane was quantified by cell uptake of the small (4 kDa) FITC-labeled Dextran. Fluorescence intensity changes due to internalized FITC-labeled Dextran and auto fluorescence of over 20,000 cells per sample was measured using a fluorescence-activated cell sorting (FACS) Calibur system (BD Biosciences, New Jersey) with the fluorescence channel. The forward scatter vs. side scatter density plots were also obtained and careful gating was applied to segment populations of live cells for further analysis, i.e. by excluding cell debris and cell-doublets. For each pre-treatment (and growth media) condition 4 measurements were collected: strain-loaded cells with or without added dextran, and unstrained control cells with or without dextran; samples without dextran provide a control for native cell auto-fluorescence (AF). The percent change in dextran uptake is calculated by the mean value of the strained sample relative to the unstrained samples, after subtracting the auto-fluorescence for each.

### Membrane Permeability Experiment Results

Using murine fibroblast (NIH3T3) and myoblast (C2C12) cell lines as a models, respectively, for superficial and deep tissue damage, the effect of NaPy on the cells' ability to maintain the integrity of its plasma membrane under damaging strains was demonstrated. The cells were pretreated with varying levels of NaPy for 4 hours and then applied sustained, radial stretching at damage-level of 12% strain for 3 hours (FIG. 2). Membrane permeability was measured by the uptake of a small (4 kDa) fluorescent Dextran, comparing pre-treated cells with untreated cells and unstrained negative controls; uptake was quantified via FACS.

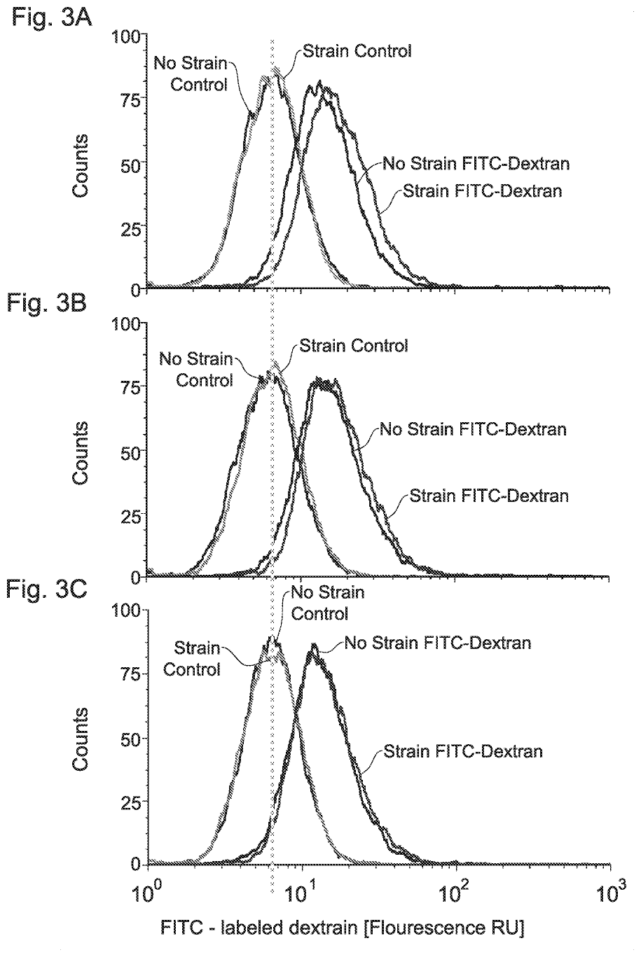

FIG. 3 shows representative results of effects of 12% stretching (strain-loading) and NaPy pre-treatment concentration (0/1/5 mM) on the plasma membrane permeability. It was noted that the auto-fluorescence of strained and unstrained controls (without dextran marker) are indistinguishable; this is demonstrated in the C2C12, yet occurs consistently in both cell types and under both growth media NaPy concentrations (0/1 mM). Auto-fluorescence is an indirect indication of morpho-functionality of cells, hence, at this early stage of cell damage the membranes may become porated and homeostasis disrupted, yet the cell structure is still intact. Uptake of FITC-labeled small-molecule dextran was used to identify changes in membrane permeability; the diameter of the 4 kDa dextran marker has previously been shown to be 2.6 nm. Unstrained cells exhibit a measurable natural uptake of the small molecule that is similar under all NaPy pre-treatment concentrations. The natural uptake in unstrained cells serves as the baseline signal, related to natural homeostatic conditions. An increased dextran internalization (higher signal) into cells that are strained to damaging-levels (12%) was observed, especially without NaPy pre-treatment. When cells are pre-treated with increasing concentrations of NaPy, proportionally reduced uptake (a left-shift of the curves) was observed into the strained cells bringing the measured uptake closer to the respective baseline. Specifically, when no NaPy pretreatment was added prior to straining (0 mM NaPy in FIG. 2) an increased uptake of dextran (FIG. 3A) was observed, as compared to NaPy pre-treated cells (FIGS. 3B and C). To evaluate effects of the experimental parameters (i.e. cell type, NaPy in growth media, NaPy pretreatment concentration) on changes in membrane permeability following strain-loading the fluorescent dextran uptake was normalized (see methods) relative to the baseline while subtracting the auto-fluorescence (blank) for strained and unstrained samples.

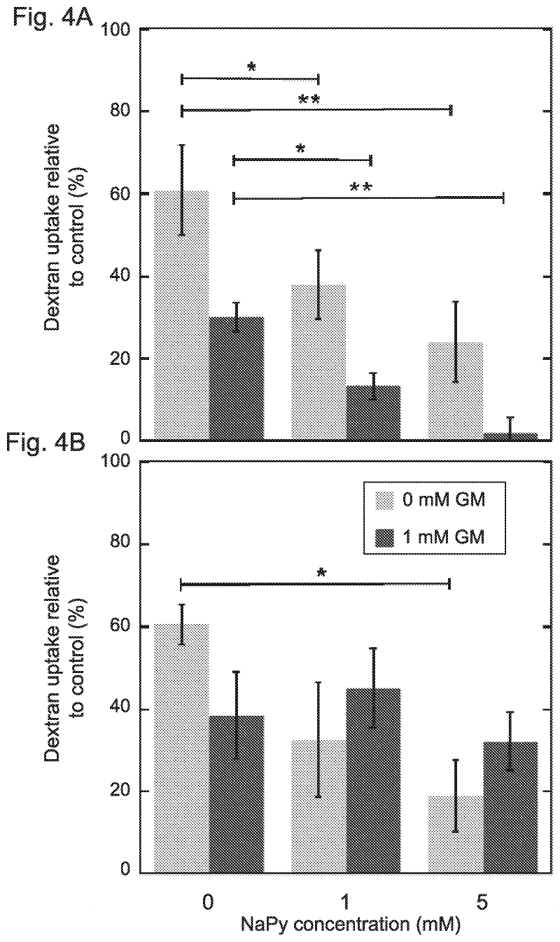

Pre-treatment of fibroblasts or myoblasts with sodium pyruvate reduces, in a concentration-dependent manner, the strain-damage induced membrane poration and permeability, which are precursors to loss of cell homeostasis and death (FIG. 4). In the C2C12 myoblasts, a reduction was observed in the cell damage-induced uptake of dextran relative to control that is proportional to the NaPy concentration (FIG. 4A); differences in dextran uptake with increased NaPy concentration were statistically significant (p<0.05). That is, with increasing NaPy concentration a reduction in the differences between the 12% strained cells and their unstrained control was noted. It should be noted that when cells have been pre-exposed to NaPy in their long-term growth media, the NaPy pre-treatment protective effect was more significant. For example, when C2C12 cells were cultured in media containing 1 mM NaPy and then pre-treated with 5 mM NaPy prior to damage application, a reduction was observed in the strain-damage induced membrane poration to insignificant levels. That is, in this condition, differences between dextran uptake of the strained cells and the unstrained control cells were indistinguishable. Thus, extended pre-exposure and pre-treatment with NaPy reduces strain-damage induced disruption of plasma membrane integrity. In the NIH3T3 fibroblasts, a similar effect of reduced damage to membrane integrity (reduced uptake relative to control) was observed with increasing concentrations of NaPy pre-treatment.

Low- and Medium-Level Stretching Combined with Sodium Pyruvate Accelerate Migration of Fibroblasts and Myoblasts

### Gap Closure Experiments

To demonstrate the combined effects of NaPy supplement concentration in the media (0, 1, or 5 mM) and low and medium levels of stretching (3-6%), combinations of both parameters were applied from the time of wounding on the rate of gap closure the time-dependent closure of small gaps was monitored. Mouse myoblast or fibroblast cells (1×10⁶ per well, previously grown media containing 1 mM NaPy) were seeded 1-3 days prior to performing a stretching experiment in a six-well (31-mm diameter) culture plate with an elastic, 0.51-mm thick, transparent and collagen-coated substrata bottom (Flexcell Inc., Burlington, N.C.). Cells were grown on the stretchable-bottom plate until a confluent monolayer had formed. The plates with cells were then mounted onto our stretching apparatus and radial stretching to tensile strains of 3% or 6% or 0% (no-stretch) control was applied Immediately following stretching, compressive deformation damage was induced in each monolayer using a rigid optic fiber (˜350 μm diameter). Cells at the center of each well were crushed, inducing an approximately circular cell-damage area with varying sizes (0.05-0.5 mm²). Media was replaced to remove any cell debris and the fresh media contained varying concentrations of sodium pyruvate: 0, 1 or 5 mM. Then the time-dependent gap closure was evaluated to reveal kinematic features The stretching apparatus was mounted in the motorized microscope stage to facilitate for time-lapse imaging of progression of the gap closure. Cell viability throughout the prolonged experiments was ensured by maintaining 37° C., 5% CO₂ and high humidity, using an incubator that enclosed the microscope (Life Imaging Services, Basel, Switzerland).

### Imaging and Analysis of Gap Closure

Time-lapse imaging was performed using a fully motorized, inverted fluorescence microscope (Olympus IX81, Tokyo, Japan) with a custom MATLAB 2012b (The MathWorks, Natick, Mass.) graphical user interface (GUI) module to automatically control lens positioning and collect images of the gap every 10 minutes for up to 24 hours. Images were taken using an XR Mega-10AWCL camera (Stanford Photonics Inc., Palo Alto, Calif.), using a 10×/NA 0.3 long working-distance, air immersion objective lens at a final magnification of 646.8 nm/pixel. A custom algorithm in MATLAB 2012b was used to automatically analyze the time-progression images of the gap area closure and quantify cell migration and gap closure progression, as described in Topman et al. (Micron 51:9-12; 2012); Toume et al. Int Wound J 14:698-703; 2017). Briefly, the time-dependent area was fit to a Richard's function, which is an asymmetric sigmoid, by minimizing the mean squared error. Using the fitted curve, the maximum migration rate was calculated, which is the maximal slope of the area vs. time, Richards fit. The normalized maximum migration rate was obtained by dividing the maximum migration rate by the initial gap area. In addition, the time for 90% gap area closure, which is indicative of the end of the en masse cell migration regime was obtained (Topman et al. 2012; Med Eng Phys 34:225-232).

### Statistical Testing in Gap Closure Experiments

Results of the different conditions were compared using a two-way analysis of variance (ANOVA) for unequal length samples; Statistical analysis was performed in MATLAB. The interaction parameter was found significant, thus the interactions between the sodium pyruvate concentration and the stretching level and were important; in cases where only the interaction parameter the interaction is considered ‘simple’ by typical definitions in statistical analysis. A one-way ANOVA was also performed in conjunction with post-hoc Tukey-Kramer tests to identify statistically significant differences levels of sodium pyruvate for each level of stretching and vice versa. A P-value lower than 0.05 was considered significant.

### Results and Discussion of Gap Closure Experiments

Both tested cell types, C2C12 myoblasts and NIH3T3 fibroblasts were cultured in media with 1 mM sodium pyruvate up to the time of infliction of compressive deformation damage (micro-gap formation) and concurrent application of radial stretching. At that time, media was replaced with media containing 0, 1 or 5 mM NaPy, thus conditions are of concurrent treatment with NaPy (either at 1 or 5 mM) and of stretching following removal of NaPy (1 mM NaPy growth media replaced with 0 mM media during gap closure).

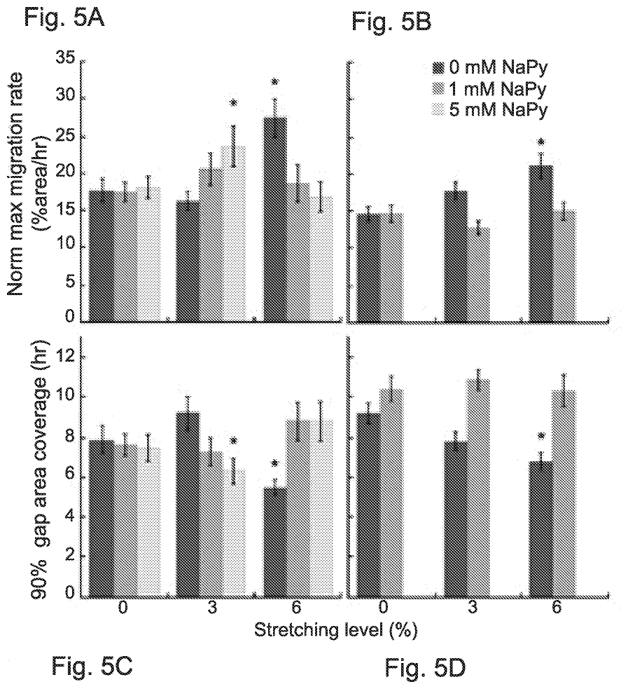

When cell monolayers are not stretched prior to wounding (i.e. 0% stretching), the gap closure rate is unaffected by the post-injury NaPy concentration (0, 1, or 5 mM) for both the myoblasts and the fibroblasts (FIG. 5). It should be noted that the migration rates of the NIH3T3 fibroblasts are generally slower, yet differences are not statistically significant.

In both myoblasts and fibroblasts, the beneficial effects of low- and mid-level stretching are highly dependent on the post-injury NaPy concentration; the cells were grown in 1 mM NaPy up to the injury. When NaPy is supplemented (1 or 5 mM) post-injury, gap closure is significantly accelerated specifically when combined with low-level stretching (3%) in damaged myoblast monolayers (FIG. 5). The normalized gap-closure rate of the myoblasts increases from 16.3% area/hr to 20.5 and 23.7 (being a 26% and 46% increase), respectively, when 1 or 5 mM NaPy are supplemented together with 3% stretching; the time to reach 90% gap coverage decreases from 9.2 hrs, respectively, to 7.3 and 6.3 hrs (being a 21% and 31% decrease). In parallel, it was noted that if NaPy is lacking post-injury the applied stretching can compensate and accelerate gap closure in both cell types. That is, in cells that had previously been exposed to NaPy, yet it is not supplemented in the post-injury medium, stretching accelerates gap closure. Specifically, the maximal migration rates of the myoblasts and the fibroblasts (FIG. 5A-B), respectively, increase from 17.7 to 27.4% area/hr and from 14.6 to 21.1% area/hr (being a 55% and 45% increase) under 6% stretching when NaPy is not supplemented post-injury. In the myoblasts, the acceleration of cells with no NaPy supplement is only statistically significant with 6% stretching. In contrast, in the fibroblasts the acceleration is proportional to the stretching level, at 3% and 6% stretching, respectively, increasing from 14.6% area/hr under 0% stretching to 17.7 and 21.1% area/hr (being a 21% and 45% increase); differences are statistically significant with p<0.05.

Specific combinations of low and medium-level stretching with exogenous NaPy supplement induced a marked increase in gap closure rate. For both the fibroblasts and myoblasts, when no NaPy supplement was provided post-injury to cells that had previously been exposed to it, the mid-level stretches (6% strain) compensated for deficiency in exogenous NaPy after injury and gap closure was accelerated in a statistically significant manner Importantly, in the myoblasts the smallest evaluated strain (3%) combined with post-injury exogenous NaPy supplement successfully accelerated gap closure in a concentration dependent and statistically significant manner As noted, in the fibroblasts, the pre-damage exposure to low levels of NaPy (1 mM) was sufficient, when combined with low- or medium-level stretching (respectively, 3% or 6%) to accelerate gap closure in a statistically significant manner Lower stretching levels are preferable in the long term, as they reduce the risk for mechanical damage. Hence, the “sweet spot” combination of low stretching levels (e.g. the 3% used here) with low levels of exogenous NaPy (1 mM) supplement provides an optimized treatment protocol for gap closure acceleration.

In the experiments performed in the gap closure study, the focus is on microscale gaps that may be used as a simplified model for damage repair or, for example, represent the actual scale of damage caused during initiation of pressure ulcers (pressure injuries), given that the initial stage of pressure ulcer formation includes death of small groups of cells. In this context, the sites of mechanical deformation-induced damage can be in some cases be foreseen and depend on patient anatomy and length of immobility period, such as when a person is anesthetized at a certain body posture in preparation for surgery. In our experiment, the stretching and the media replacement are applied together with the injury, effectively performing the “wounding” and the “treatment” (cleaning of cell debris and related signaling molecules, NaPy supplement, applied stretching) at the same time. For the example of pressure ulcer initiation, in performing the stretching immediately prior to cell death, a condition of pre-treatment is in fact simulated prior to or immediately following initial cell damage, in a preventative approach; closure of small gaps will prevent the cascade of further damage development.

### Measuring Subepidermal Moisture to Detect Pressure Injury

Cell death at the onset of a PU triggers inflammatory processes that as a side-effect lead to blood plasma fluids escape from the vasculature, resulting in microscopic onset of edema. The volume of the fluids builds up gradually in the tissue, eventually forming edema. As more immune cells are recruited to the cell death site, the process that begins microscopically progresses to a larger scale. A commercial technology to measure the change in fluid content, i.e. the subepidermal moisture (SEM) has recently been introduced in the form of a SEM Scanner (Bruin Biometrics Europe) which provides a clinically indicative biophysical biomarker for early stages of PU formation. The SEM scanner is a hand-held device sensitive to changes in the biocapacitance of the affected soft tissues, or their resistance to transmission of non-damaging electrical fields. Specifically, gradual accumulation of fluids in tissues make the tissues progressively less resistant to electrical fields, and hence the relative permittivity (dielectric constant) of the tissues increase from that of the health tissues towards that of water. The SEM Scanner is sensitive to small changes in the amounts of extracellular fluids, and provides standardized, objective and quantitative measures for detecting changes associated with the edema build-up as a reaction to the immune response to the death of the first cells. There is a substantial volume of published literature demonstrating that the SEM Scanner can detect subdermal tissue damage 3-10 days before it is visible to the naked eye (See for example: (a) Bates-Jensen B M, McCreath H E, Nakagami G, Patlan A. Subepidermal moisture detection of heel pressure injury: The pressure ulcer detection study outcomes. Int Wound J. 2018 April; 15(2):297-309; (b) Bates-Jensen B M, McCreath H E, Patlan A. Subepidermal moisture detection of pressure induced tissue damage on the trunk: The pressure ulcer detection study outcomes. Wound Repair Regen. 2017 May; 25(3):502-511; (c) O'Brien G, Moore Z, Patton D, O'Connor T. The relationship between nurses assessment of early pressure ulcer damage and sub epidermal moisture measurement: A prospective explorative study. J Tissue Viability. 2018 in press, available online, doi: 10.1016/j.jtv.2018.06.004; (d) Gefen A, Gershon S. An Observational, Prospective Cohort Pilot Study to Compare the Use of Subepidermal Moisture Measurements Versus Ultrasound and Visual Skin Assessments for Early Detection of Pressure Injury. Ostomy Wound Manage. 2018 September; 64(9):12-27).

### Computer Simulations in Human Anatomy:

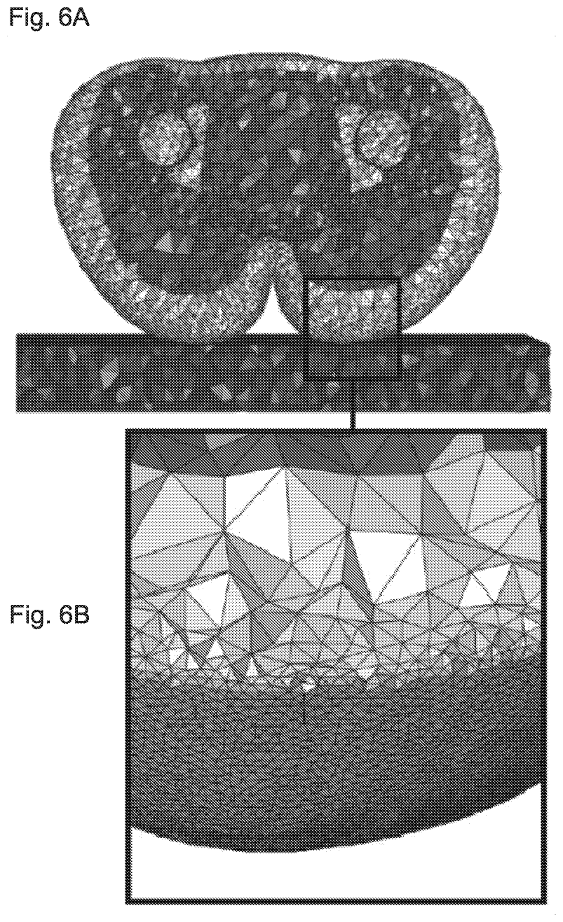

A three-dimensional finite element (FE) model of the human anatomy was developed (FIG. 6), which considers the ‘microenvironment’ (temperature and humidity) and applied simulated-deformation (initially compression) due to mattresses. An MRI based buttocks FE model was used and several predictive tools were incorporated to simulate how microclimate affects tissue biomechanically and biothermally in macroscale when in the patient is in bed.

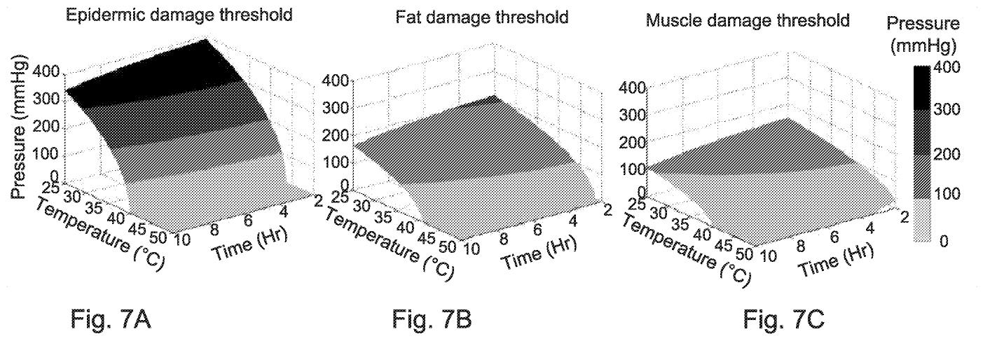

The results of this model are temperature, strain and damage score maps of the 3D buttocks model under different conditions, which are shown in FIG. 5. A higher damage threshold on the epidermal layer as compared to fat and muscle was observed. For all three tissue types, the threshold for damage was temperature dependent, exponentially increasing over 40° C. (FIG. 7).

Delivery of Sodium Pyruvate (NaPy) from an Active Dressing to Protect Sacral Skin and Underlying Tissues

Four finite element computational model variants of the sacral skin were developed, representing four levels of skin roughness. These model variants were used to investigate how prophylactic dressings, pre-loaded with NaPy, can be used for transdermal delivery of NaPy, for improving skin and subcutaneous tissue tolerance to sustained deformation exposures and hence, tissue resistance to PU development. For this purpose, application of such novel dressings loaded with a known NaPy concentration was modeled and monitored simulated free NaPy diffusion into the skin for a period of 16 hours.

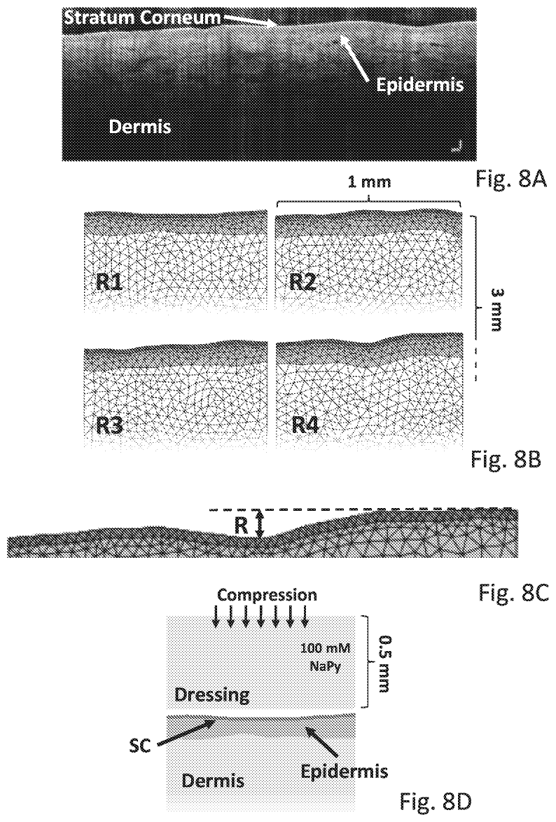

Optical coherence tomography (OCT) images of the sacral skin were used to generate four two-dimensional anatomical skin models, representing different levels of skin roughness (FIGS. 8A-B). Two-dimensional images were acquired with a scan length of approximately 5 mm, a lateral resolution of up to 8 μm, and a maximum penetration depth of ˜1.5 mm Segmentation software (the ScanIP module of Simpleware®) was used to segment the different layers of the skin. A roughness criterion (R) was set as the maximal vertical distance between the top of the highest fold and the bottom of the lowest valley of the stratum corneum (SC), and the four areas of interest were chosen so that R₁=20 μm, R₂=30 μm, R₃=40 μm, and R₄=50 μm (FIGS. 8B-C; R₁ and R₂ are from the same subject, and likewise, R₃ and R₄ are from the same other individual). Then, a constant minimal thickness was assigned to the anatomical model variants so that each of the variants was 1×3×0.05 mm and included the SC, epidermis and dermis layers (FIG. 8B). Next, a geometrical representation of a flat dressing was added to each of the model variants, and positioned as close as possible to the rough SC surface (FIG. 8D).

The dressing and skin layers were considered as biphasic-solute materials in order to be able to simulate the coupled structural response of skin to the (bodyweight) loading and the diffusion of the NaPy released from the dressing as it compresses onto the skin (under the bodyweight forces). Physical and diffusional properties of the skin layers, dressing and NaPy molecules were adopted from the literature. Specifically, NaPy molecules were assigned a molecular weight of 110 g/mol and a neutral chemical charge. Solubility, diffusivity and free diffusivity of NaPy molecules in the aqueous phase of the dressing and tissues were considered isotropic and set at 100 mg/mL, 0.0005 mm²/s and 0.001 mm²/s, respectively.

The dressing and skin layers were assigned a solid volume fraction of 0.2 and constant isotropic permeability characteristics as follows. Dermal permeability coefficient (Kₚ) of NaPy was calculated using:

log Kₚ=−2.72+0.71·(log Ko/W)−0.0061·(MW)

where Ko/W=−5.05 and MW=110.04 g/mol.

Since the skin barrier properties are mostly attributed to the SC layer, and given that transepidermal water loss (TEWL) increases 20-fold when the SC is removed, the permeability coefficients of the epidermis and dermis were set as 20-times greater than that of the SC. The final Kₚ values of skin were set at 2.9322·10⁻¹⁰ mm/s for the SC and 5.86445·10⁻⁹ mm/s for the epidermis and dermis. The Kₚ of the dressing was set at 0.001 mm/s.

Constitutive laws and mechanical properties of the model components were also adopted from the literature. Specifically, the dressing material was assumed to be isotropic linear-elastic material with an elastic modulus of 19 kPa and a Poisson's ratio of 0.3. The skin layers were assumed to be nearly incompressible (Poisson's ratio of 0.49), non-linear isotropic materials with their large deformation behavior described using an uncoupled Neo-Hookean material model with a strain energy density (SED) function W:

$$W = \frac{{G}_{ins}}{2} ( {λ}_{1}^{2} + {λ}_{2}^{2} + {λ}_{3}^{2} - 3 ) + \frac{1}{2} {K ( ln J )}^{2} \qquad ( 1 )$$

where Gᵢₙₛ is the instantaneous shear modulus, λᵢ (i=1, 2, 3) are the principal stretch ratios, K is the bulk modulus and J=det(F) where F is the deformation gradient tensor. The instantaneous shear moduli assigned to the skin layers were 839 kPa, 352 kPa and 7.55 kPa for the SC, epidermis and dermis, respectively.

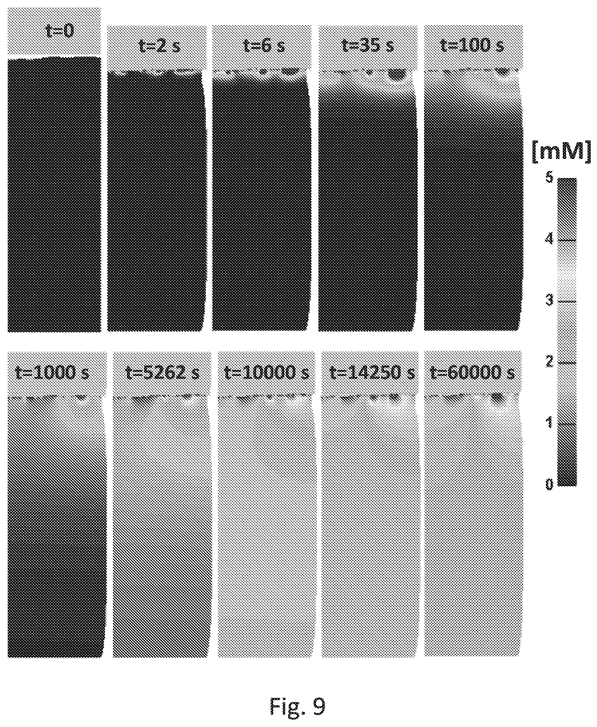

Boundary conditions were chosen to simulate the application of the novel prophylactic sacral dressing loaded with NaPy in a thin-slice model configuration. The dressing was assigned an initial homogenous concentration of 100 mM and the top surface of the dressing was held at a constant NaPy concentration throughout the simulation, which represented the intra-dressing NaPy reservoir. The front, back, bottom and side planes of the dressing and skin layers were assigned zero flux across them, and biphasic-solute contact was defined only between the bottom of the dressing and top of the SC, so that NaPy molecules may only leave the dressing into the skin, where contact is established (but not return to the dressing). The front, back and left planes of the dressing and skin were fixed for perpendicular displacements, while the right surfaces were released to allow adequate convergence of the numerical simulation and relaxation of the osmotic stresses during the diffusional response. The dressing was then lowered by 0.2 mm over two seconds, until contact was established between the dressing and the SC, and NaPy molecules were allowed to translate by diffusion from the dressing into the skin, over a period of 16 hours of simulated application (FIG. 9).

Meshing the model variants was again performed by means of the ScanlP® module of Simpleware®, using 4-node linear tetrahedral elements (FIGS. 8B-C). Each model included approximately 1,700 elements describing the dressing, 1,550 elements describing the SC, 1,840 elements describing the epidermis and 8,250 elements describing the dermis.

Simulations were set up in PreView of FEBio (Ver. 1.19), analyzed using the Pardiso linear solver of FEBio (http://mrl.sci.utah.edu/software/febio) (Ver. 2.5.0) in its biphasic-solute transient mode, and post-processed using PostView (Ver. 1.10). A 64-bit Windows 8-based workstation with 2×Intel Xeon E5-2620 2.00 GHz CPU and 64 GB of RAM was used for solving the coupled structural-diffusion problems of the NaPy release from a prophylactic dressing.

The transient average effective NaPy concentrations in the SC and epidermis layers together, and at depths of 1 mm, 2 mm and 3 mm into the dermis were measured for the four examined levels of OCT-measured skin roughness. The times until steady-state has been reached were additionally calculated for each model variant and each examined depth of skin. The time required for convergence of a run was defined as the time from the simulated application of the dressing to the first time point where the NaPy concentration level at the examined depth did not change by more than ±10% with respect to the plateau value.

An example time course of the NaPy concentration as the substance enters the skin is shown in FIG. 9. During the vertical displacement of the dressing, contact area between the dressing and the SC is established and NaPy molecules begin to diffuse from the dressing into the skin. Diffusion persists until steady-state is reached and NaPy concentration stabilizes, as expected. The average dermal NaPy flux stabilized after ˜5 minutes at 0.35 nmol/cm²·h.

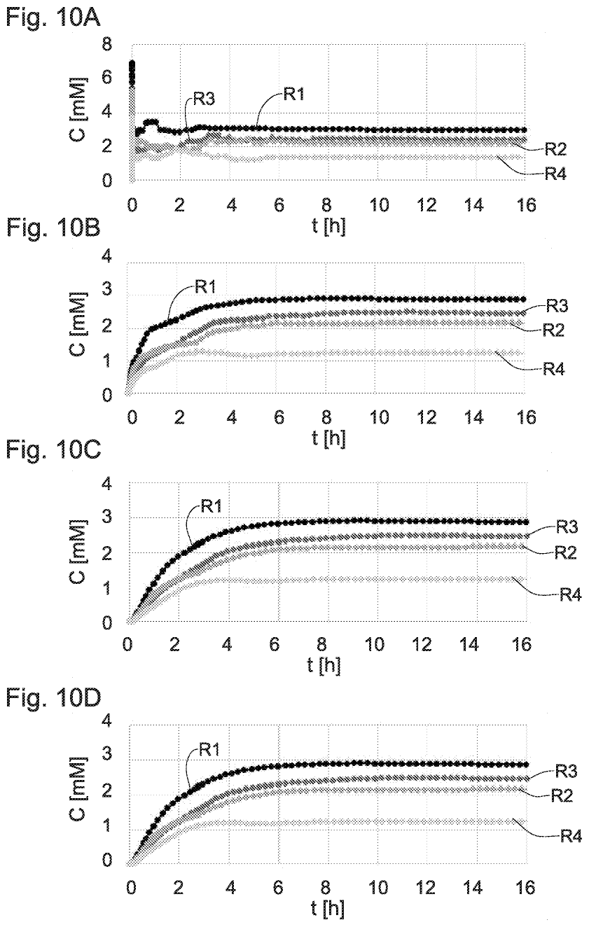

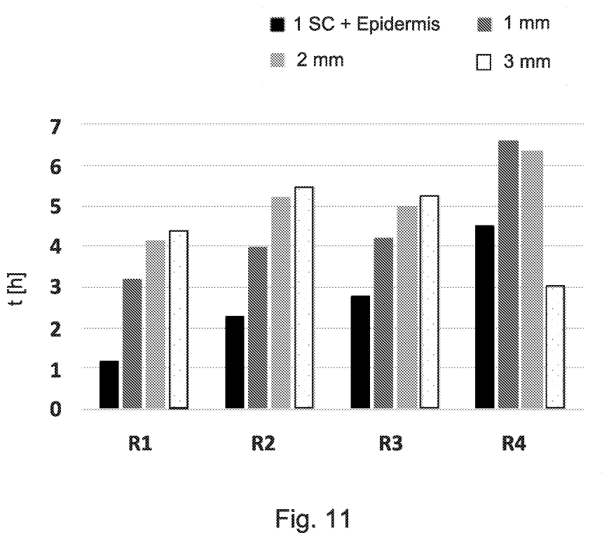

NaPy concentrations in the SC and epidermis layers increased rapidly in all the model variants, and has peaked at 6.92 mM, 4.85 mM, 5.3 mM and 3.74 mM in model variants R₁, R₂, R₃ and R_{4,} respectively, 7-9 seconds post application of the dressing (FIG. 10A). The NaPy concentrations stabilized after 1.15-4.5 hours at 2.98 mM, 2.45 mM, 2.23 mM and 1.34 mM in model variants R₁, R₂, R₃ and R₄, respectively (FIGS. 10A and 11), that is a coefficient of variation (COV) of 30.4% which reflects the micro-anatomical variability and its impact on the variability of the individualized diffusion responses. Across all the examined dermis depths, NaPy concentrations stabilized after 3-6.6 hours (for all micro-anatomies), at values of 2.88 mM, 2.45 mM, 2.16 mM and 1.24 mM in model variants R₁, R₂, R₃ and R₄, respectively (FIGS. 10A and 11), which yields a COV of 31.8% (again pointing to the extent of variability in individual diffusion responses). Correspondingly, it was found that the relative contact areas between the dressing and the SC reached 54.1%, 51.1%, 44.6% and 28.7%, in model variants R₁, R₂, R₃ and R₄, respectively. The latter demonstrates the potential variability in dressing-skin contact conditions, which is a derivative of the individual skin roughness, being yet another factor that influences the individual diffusion pattern of NaPy into the skin.

Overall, in all but one model variant, NaPy concentrations stabilized faster in the more superficial layers of the skin and slower deeper within the dermis, which could be foreseen given that release is from the NaPy reservoir within the dressing (FIG. 11). For example, in model variant R₂, NaPy concentration reached 90% of its final value after 2.28, 3.96, 5.2 and 5.47 hours, in the SC/epidermis, and 1, 2, and 3 mm deep into the dermis, respectively. Additionally, in all but one examined depths, NaPy concentrations stabilized faster in the model variants that represented smoother skin surfaces. For example, at 2 mm into the dermis, steady-state has been reached after 4.13, 5.2, 4.98 and 6.3 hours in model variants R₁, R₂, R₃ and R₄, respectively where R₁, R₂, R₃ are the smoother skins. As mentioned already, this behavior originates from the quality of attachment of the dressing onto the skin, which typically has less contact area for a more rough skin surface (such as in an aged, wrinkled skin).

In summary, a multiple OCT-based FE computational model variants was developed and used in order to evaluate the transdermal delivery capacities of novel NaPy-loaded prophylactic sacral dressings in different individuals (patent pending by co-inventor AG). Further, effects of skin roughness were studied on the resulting diffusional response of the NaPy released from the dressing. A steady state NaPy concentrations was found in the dermis, being 1.25-3% of the concentration loaded in the applied dressing, which is in good agreement with published data regarding the absorbance capacity of dermal tissues. Steady state concentrations also found within the dermis were correlated with the relative contact area between the dressing and the SC, which is a direct outcome of the skin roughness level. This effect was to be expected since the greater the contact area available for diffusion of NaPy, the greater the NaPy flux is across the contact area, and the greater its resulted concentrations in deeper tissue layers. Nevertheless, it should be noted that in real-world conditions, the skin roughness under the dressing will likely decrease as the skin temperature and (the associated) level of SC hydration are expected to increase. Altogether, a smoother, warmer and more moist skin under the dressing (compared to bare skin) would result in elevated dermal permeation and hence, increased intra-dermal NaPy diffusion with respect to our present modeling predictions which did not consider these complex skin-dressing interactions at this stage.

In general, the diffusion of molecules through the skin is mostly attributed to the barrier properties of the SC, and also, strongly, on the size of the diffusing molecule, its hydrophilic or hydrophobic nature, and the applied concentration gradient. The permeability of intact human skin is significantly decreased for diffusants with a molecular weight (MW) above 500 Daltons. The SC, which is only a few micrometers thick, consists of apoptotic keratinocytes surrounded by keratin-rich lipid bilayers. This means that small hydrophilic molecules penetrate through the SC via an intracellular route, or through the hair follicle/sweat glands openings in the SC. Hence, the level of hydration of the SC has a substantial effect on the diffusivity of small hydrophilic molecules, such as NaPy, with greater diffusivity as the hydration level increases. Furthermore, as blood flow in the dermis increases, transport of small hydrophilic molecules increases as well and diffusion to deeper tissues wanes down. The hydration level of the SC as well as the blood flow in the dermis should therefore be considered in future modeling of transdermal delivery from prophylactic dressings.

In the medical literature, there are only sparse data regarding the permeability of the SC to NaPy. However, pyruvic acid is commonly used topically on the skin (e.g. in the treatment of acne), with obvious potency in deeper skin layers, and hence could be assumed that NaPy is able to penetrate the SC spontaneously and in a similar way. Furthermore, the results of the simulations agree with those obtained by others who examined ex-vivo transdermal delivery of various topical analgesic medications, using Franz Diffusion Cells. Early studies found that the transdermal flux of Diclofenac Sodium which is a similar sodium salt with a MW of 318 g/mol that has been applied on the skin using a 3%-cream reached 0.321-0.943 nmol/cm²·h after 24 hours. It was also shown that a total of 0.846-1.96% of the applied diclofenac sodium was absorbed in the skin after 48 hours.

NaPy is a relatively small molecule (e.g. compared to pyruvate acid) and hence it diffuses freely in the aqueous phase of soft tissues. For example, the corneal penetration of NaPy has been studied in living human eyes 2 hours prior to extraction of the corneal tissue due to cataract surgery. The level of NaPy in control tissue samples of patients who did not receive NaPy eye drops was only 0.145+/−0.06 mM (which reflects the natural, baseline corneal NaPy levels, whereas in the group given the NaPy eye drops, it increased to approximately 0.35-0.525 mM. These findings are in good agreement with the results of the present simulations with regard to timeframes for plateau of diffusion and concentration levels at the target tissues, however, the use of prophylactic dressings, unlike the application of eye drops or creams, allows for continuous administration of NaPy molecules. Accordingly, release of NaPy from a prophylactic dressing is able to induce a relatively constant concentration in the target soft tissues over time, which is a considerable advantage when the goal is PUP for an estimated specific time frame, such as during (a certain, known type of) surgery. Additionally, the few hours needed to achieve a steady state potent NaPy concentration in our modeling, make a reasonable timeframe for applying such NaPy-loaded sacral prophylactic dressings prior to a planned, scheduled surgery. For example, if the surgery is to be performed in a supine patient, a NaPy-releasing prophylactic sacral dressing can be applied approximately 4 hours prior to anesthesia (based on the data shown in FIG. 10) to enhance sacral soft tissues tolerance to the sustained deformations caused by bodyweight forces.

In conclusion, NaPy-releasing prophylactic dressings is capable of improving soft tissue tolerance to sustained deformations. The time needed to achieve steady-state NaPy concentrations in the dermis was approximately 4 hours, which makes the protective effect of such dressings applicable in preparing a patient for surgery, or for use in intensive care units. Individual skin roughness might theoretically affect the resulting NaPy concentration in the dermis, considering microclimate and skin barrier alternations that likely occur under the dressing.

1. A method for preventing pressure ulcers or injuries in a subject, the method comprising administering at least one pyruvate compound to a subject to thereby treat microscopic damage to a skin region or tissue region due to an intense or prolonged pressure or pressure in combination with shear, wherein the method excludes treatment of existing or visible pressure injuries.

2. The method according to claim 1, wherein said compound is pyruvic acid or an ester, amide or salt thereof.

3. The method according to claim 1, wherein said compound is pyruvic acid.

4. (canceled)

5. The method according to claim 42, wherein the ester is selected from alkyl esters and substituted forms thereof, wherein the alkyl ester is optionally derived from alcohols having between 1 and 10 carbon atoms.

6-7. (canceled)

8. The method according to claim 4, wherein the pyruvate ester is methyl pyruvate, ethyl pyruvate or propyl pyruvate.

9. (canceled)

10. The method according to claim 2, wherein the amide is derived from an amine selected from ammonia, mono-alkyl amine, di-alkyl amine and tri-alkyl amine, wherein the alkyl comprises from 1 to 10 carbon atoms.

11. The method according to claim 2, wherein said compound is a salt of pyruvic acid.

12. The method according to claim 11, wherein the salt is a base-addition salt derived from an organic or inorganic base.

13. The method according to claim 11, wherein the salt is selected from sodium pyruvate, calcium pyruvate, zinc pyruvate, lithium pyruvate, potassium pyruvate, magnesium pyruvate and manganese pyruvate.

14. The method according to claim 13, wherein the salt is sodium pyruvate.

15-29. (canceled)

30. The method according to claim 1, wherein the at least one pyruvate compound is administered with a low- to medium-level of a stretching force or deformation or strain to the skin region or to the deeper tissue region or to the organ of the subject, wherein said administration is prior to, subsequently to, concomitantly with or after administration of the pyruvate compound.

31-48. (canceled)

49. A device or an article comprising a releasable pyruvate compound, the device or article being configured and operable for prevention of pressure ulcers, and selected from a dressing, a pad, a bandage, an underwear, a diaper, a plaster, a patch, a mattress, a cushion, a seating surface, a surgical table, an examination table, a continuous positive airway pressure mask, an oxygen mask, a spinal board, a cervical collar, a pulse oximeter, a catheter, wiring, an electrode, a tracheostomy device, a nasogastric tube, an endotracheal tube, compression stockings, a cast, a positioner, a heel boot, a headrest, a footrest, a lying surface and a cover.

50. A device for inducing deformation of a skin region of a subject, the device comprising:
an outer, non-deformable or minimally-deformable frame element, having at least one first skin-contacting surface configured to adhere to a first skin region of the subject;
at least one inner, elastically, visco-elastically or plastically deformable element, having at least one second skin-contacting surface that is configured to adhere to a second skin region of the subject; wherein the second skin region is within boundaries formed by the outer frame element; and
a member disposed between and connecting said outer element and inner element; wherein the member is elastic, viscoelastic or plastic;
wherein the elastic, viscoelastic or plastic member is configured to be selectively deformable, thereby causing elastic, viscoelastic or plastic deformation of the at least one inner element and said second skin region.

51-52. (canceled)

53. The device according to claim 50, wherein the inner frame is concentric with the outer frame such that the inner frame is in contact with a skin region that is contained within boundaries formed by the outer ring.

54. The device according to claim 50, wherein the elastic, viscoelastic of plastic member is configured to selectively deform upon application of at least one of a mechanical force, a magnetic force, a magneto-mechanical force, an electrical input, a chemical reaction, a temperature change and at least one skin-environmental condition or any combination thereof.

55. The device according to claim 54, wherein the elastic, viscoelastic, or plastic member is induced to deform in response to at least one skin-environmental condition selected from exposure to air, skin, body-heat, perspiration, pH changes, or any combination thereof.

56. The device according to claim 50, wherein the selective deformation of the elastic, viscoelastic or plastic member is user-induced.

57. The device according to claim 56, wherein the user-induced selective deformation is manual, automatic, responsive or semi-automatic.

58. The device according to claim 50, being for use in combination with a prophylactic dressings, bandages, patches, garments, underwear or gels or for use in combination with administration of at least one pyruvate.

59. The device according to claim 58, wherein the at least one pyruvate is contained within any skin-contacting element or region of the device.
